SEQUENCE LISTING (3 sequences)

SEQ ID NO: 1 - protein, 1027 aa, Artificial Sequence
  Purified recombinant hNLRP3 (6His-SUMO-TEV- NLRP3 [125-1036])
MGHHHHHHGSLQDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGENLYFQGSRISICKMKKDYRKKYRKYVRSRFQCIEDRNARLGESVSLNKRYTRLRLIKEHRSQQEREQELLAIGKTKTCESPVSPIKMELLFDPDDEHSEPVHTVVFQGAAGIGKTILARKMMLDWASGTLYQDRFDYLFYIHCREVSLVTQRSLGDLIMSCCPDPNPPIHKIVRKPSRILFLMDGFDELQGAFDEHIGPLCTDWQKAERGDILLSSLIRKKLLPEASLLITTRPVALEKLQHLLDHPRHVEILGFSEAKRKEYFFKYFSDEAQARAAFSLIQENEVLFTMCFIPLVCWIVCTGLKQQMESGKSLAQTSKTTTAVYVFFLSSLLQPRGGSQEHGLCAHLWGLCSLAADGIWNQKILFEESDLRNHGLQKADVSAFLRMNLFQKEVDCEKFYSFIHMTFQEFFAAMYYLLEEEKEGRTNVPGSRLKLPSRDVTVLLENYGKFEKGYLIFVVRFLFGLVNQERTSYLEKKLSCKISQQIRLELLKWIEVKAKAKKLQIQPSQLELFYCLYEMQEEDFVQRAMDYFPKIEINLSTRMDHMVSSFCIENCHRVESLSLGFLHNMPKEEEEEEKEGRHLDMVQCVLPSSSHAACSHGLVNSHLTSSFCRGLFSVLSTSQSLTELDLSDNSLGDPGMRVLCETLQHPGCNIRRLWLGRCGLSHECCFDISLVLSSNQKLVELDLSDNALGDFGIRLLCVGLKHLLCNLKKLWLVSCCLTSACCQDLASVLSTSHSLTRLYVGENALGDSGVAILCEKAKNPQCNLQKLGLVNSGLTSVCCSALSSVLSTNQNLTHLYLRGNTLGDKGIKLLCEGLLHPDCKLQVLELDNCNLTSHCCWDLSTLLTSSQSLRKLSLGNNDLGDLGVMMFCEVLKQQSCLLQNLGLSEMYFNYETKSALETLQEEKPELTVVFEPSW

SEQ ID NO: 2 - protein, 116 aa, Artificial Sequence
  6His-SUMO-TEV tag
MGHHHHHHGSLQDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGENLYFQGS

SEQ ID NO: 3 - protein, 10 aa, Artificial Sequence
  Modified hNLRP3 peptide
TCESPVSPIK

(19) United States
(12) Patent Application Publication
(10) Pub. No.: US 2022/0163539 A1
(43) Pub. Date: May 26, 2022

(54) NLRP3 INFLAMMASOME INHIBITION

(71) Applicant: Inflazome Limited, Dublin 2 (IE)

(72) Inventors: Matthew COOPER, Cambridge (GB); Angus MACLEOD, Cambridge (GB); Reena HALAI, Cambridge (GB); Jimmy VAN WILTENBURG, Groningen (NL)

(73) Assignee: Inflazome Limited, Dublin 2 (IE)

(21) Appl. No.: 17/601,912

(22) PCT Filed: Apr. 11, 2020
(86) PCT No.: PCT/EP2020/060356

§ 371 Date: Oct. 6, 2021

(30) Foreign Application Priority Data
Apr. 12, 2019 (GB) 1905265.3

(51) Int. Cl. G01N 33/68; C07K 14/705; G16B 20/30

(52) U.S. Cl. CPC G01N 33/6893; G01N 2333/705; G16B 20/30; C07K 14/705

(57) ABSTRACT

The present invention relates to a binding site of the NLRP₃ inflammasome. The present invention further relates to a method of and a compound for use in inhibiting NLRP₃ activation and treating a disease, disorder or condition responsive to NLRP₃ inhibition. The present invention further relates to a method of reducing cellular or mitochondrial Reactive Oxygen Species (ROS) by inhibiting NLRP₃ activation. The present invention further relates to a method of screening a compound to determine the extent of binding of the compound to the binding site of the NLRP₃ inflammasome, and to a compound identified by such a screening method.

### FIELD OF THE INVENTION

The present invention relates to a binding site of the NLRP3 inflammasome. The present invention further relates to a method of and a compound for use in inhibiting NLRP3 activation and treating a disease, disorder or condition responsive to NLRP3 inhibition. The present invention further relates to a method of reducing cellular or mitochondrial Reactive Oxygen Species (ROS) by inhibiting NLRP3 activation. The present invention further relates to a method of screening a compound to determine the extent of binding of the compound to the binding site of the NLRP3 inflammasome, and to a compound identified by such a screening method.

### BACKGROUND OF THE INVENTION

Inflammasomes are responsible for the activation of inflammatory responses. The NOD-like receptor (NLR) family, pyrin domain-containing protein 3 (NLRP3) inflammasome is a component of the inflammatory process, and its aberrant activity is pathogenic in inherited disorders such as cryopyrin-associated periodic syndromes (CAPS) and complex diseases such as multiple sclerosis, type 2 diabetes, Alzheimer's disease and atherosclerosis.

NLRP3 is an intracellular signalling molecule that senses many pathogen-derived, environmental and host-derived factors. Upon activation, NLRP3 binds to apoptosis-associated speck-like protein containing a caspase activation and recruitment domain (ASC). ASC then polymerises to form a large aggregate known as an ASC speck. Polymerised ASC in turn interacts with the cysteine protease caspase-1 to form a complex termed the inflammasome. This results in the activation of caspase-1, which cleaves the precursor forms of the proinflammatory cytokines IL-1β and IL-18 (termed pro-IL-1β and pro-IL-18 respectively) to thereby activate these cytokines. Caspase-1 also mediates a type of inflammatory cell death known as pyroptosis. The ASC speck can also recruit and activate caspase-8, which can process pro-IL-1β and pro-IL-18 and trigger apoptotic cell death.

Caspase-1 cleaves pro-IL-1β and pro-IL-18 to their active forms, which are secreted from the cell. Active caspase-1 also cleaves gasdermin-D to trigger pyroptosis. Through its control of the pyroptotic cell death pathway, caspase-1 also mediates the release of alarmin molecules such as IL-33 and high mobility group box 1 protein (HMGB1). Caspase-1 also cleaves intracellular IL-1R2 resulting in its degradation and allowing the release of IL-1α. In human cells caspase-1 may also control the processing and secretion of IL-37. A number of other caspase-1 substrates such as components of the cytoskeleton and glycolysis pathway may contribute to caspase-1-dependent inflammation.

NLRP3-dependent ASC specks are released into the extracellular environment where they can activate caspase-1, induce processing of caspase-1 substrates and propagate inflammation.

Active cytokines derived from NLRP3 inflammasome activation are important drivers of inflammation and interact with other cytokine pathways to shape the immune response to infection and injury. For example, IL-1β signalling induces the secretion of the pro-inflammatory cytokines IL-6 and TNF. IL-1β and IL-18 synergise with IL-23 to induce IL-17 production by memory CD4 Th17 cells and by γδ T cells in the absence of T cell receptor engagement. IL-18 and IL-12 also synergise to induce IFN-γ production from memory T cells and NK cells driving a Th1 response.

The inherited CAPS diseases Muckle-Wells syndrome (MWS), familial cold autoinflammatory syndrome (FCAS) and neonatal-onset multisystem inflammatory disease (NOMID) are caused by gain-of-function mutations in NLRP3, thus defining NLRP3 as a critical component of the inflammatory process. NLRP3 has also been implicated in the pathogenesis of a number of complex diseases, notably including metabolic disorders such as type 2 diabetes, atherosclerosis, obesity and gout.

A role for NLRP3 in diseases of the central nervous system is emerging, and lung diseases have also been shown to be influenced by NLRP3. Furthermore, NLRP3 has a role in the development of liver disease, kidney disease and aging. Many of these associations were defined using Nlrp3−/− mice, but there have also been insights into the specific activation of NLRP3 in these diseases. In type 2 diabetes mellitus (T2D), the deposition of islet amyloid polypeptide in the pancreas activates NLRP3 and IL-1β signalling, resulting in cell death and inflammation.

Several small molecules have been shown to inhibit the NLRP3 inflammasome. Glyburide inhibits IL-1β production at micromolar concentrations in response to the activation of NLRP3 but not NLRC4 or NLRP1. Other previously characterised weak NLRP3 inhibitors include parthenolide, 3,4-methylenedioxy-p-nitrostyrene and dimethyl sulfoxide (DMSO), although these agents have limited potency and are nonspecific.

Current treatments for NLRP3-related diseases include biologic agents that target IL-1. These are the recombinant IL-1 receptor antagonist anakinra, the neutralizing IL-1β antibody canakinumab and the soluble decoy IL-1 receptor rilonacept. These approaches have proven successful in the treatment of CAPS, and these biologic agents have been used in clinical trials for other IL-1β-associated diseases.

Some diarylsulfonylurea-containing compounds have been identified as cytokine release inhibitory drugs (CRIDs) (Perregaux et al., J Pharmacol Exp Ther, 299: 187-197, 2001). CRIDs are a class of diarylsulfonylurea-containing compounds that inhibit the post-translational processing of IL-1R. Post-translational processing of IL-1R is accompanied by activation of caspase-1 and cell death. CRIDs arrest activated monocytes so that caspase-1 remains inactive and plasma membrane latency is preserved.

Certain sulfonylurea-containing compounds are also disclosed as inhibitors of NLRP3 (see for example, Baldwin et al., J. Med. Chem., 59(5), 1691-1710, 2016; and WO 2016/131098 A1, WO 2017/129897 A1, WO 2017/140778 A1, WO 2017/184623 A1, WO 2017/184624 A1, WO 2018/015445 A1, WO 2018/136890 A1, WO 2018/215818 A1, WO 2019/008025 A1, WO 2019/008029 A1, WO 2019/034686 A1, WO 2019/034688 A1, WO 2019/034690 A1, WO 2019/034692 A1, WO 2019/034693 A1, WO 2019/034696 A1, WO 2019/034697 A1, WO 2019/043610 A1, WO 2019/092170 A1, WO 2019/092171 A1, and WO 2019/092172 A1). In addition, WO 2017/184604 A1 and WO 2019/079119 A1 disclose a number of sulfonylamide-containing compounds as inhibitors of NLRP3. Certain sulfoximine-containing compounds are also disclosed as inhibitors of NLRP3 (WO 2018/225018 A1, WO 2019/023145 A1, WO 2019/023147 A1, and WO 2019/068772 A1).

However, the exact mechanism of action of inhibitors of NLRP3 is unknown.

The biochemical and structural aspects of the ATP-binding domain in inflammasome-forming human NLRP proteins is discussed in Macdonald, J. A. et al (IUBMB Life. 2013. 65(10):851-862).

In summary, all NLRPs are, in general, characterized by an N-terminal pyrin domain, a C-terminal leucine-rich repeat and a central nucleotide-binding domain (NBD). The NBD is composed of the NACHT domain and NAD (NACHT-associated domain) regions and consists of three helical subdomains connected by linker regions. NACHT is so named because of its appearance in the neuronal apoptosis inhibitor protein ((NAIP); major histocompatibility complex class II transcription activator (CIITA); incompatibility protein locus from the fungus Podospora anserine (HET-E); and mammalian telomerase-associated proteins).

The ATP binding and hydrolysis properties of the NACHT domain are central to the classification of the NLRPs within the STAND subfamily of the ATPases associated with various cellular activities (AAA1) superfamily. The domain consists of several distinct, conserved motifs, including an Mg21 coordination loop and an ATPase-specific P-loop. Central to the domain is the presence of Walker A and Walker B motifs that distinguish NLRPs from other P-loop NTPases.

The Walker A and Walker B motifs are protein sequence motifs known to have highly conserved 3 dimensional structures.

The Walker A motif is associated with phosphate binding. The Walker B motif is a motif in most P-loop proteins situated well downstream of the A motif.

There is a need to determine how inhibitors of the NLRP3 inflammasome suppress NLRP3 activation, and to identify the NLRP3 binding site.

There is also a need to identify and provide compounds that bind to the NLRP3 binding site.

### SUMMARY OF THE INVENTION

A first aspect of the present invention provides a binding site of the NLRP3 inflammasome, wherein the binding site:
(a) is at or proximal to the Walker A and/or Walker B site of the NLRP3 inflammasome; and/or
(b) comprises one or more residues selected from Arg183, Gly229, Ile230, Gly231, Lys232, Thr233, Ile234, Gly303, Asp305, Glu306, Leu413 and His522.

In one embodiment of the first aspect of the present invention, the binding site is at or proximal to the Walker A and/or Walker B site of the NLRP3 inflammasome. In one embodiment, the binding site is at or proximal to the Walker A site of the NLRP3 inflammasome.

For the purposes of the present application, the term “proximal” means less than 10 Å, preferably less than 5 Å.

In one embodiment of the first aspect of the present invention, the binding site comprises 2 or more (or 3 or more, or 4 or more, or 5 or more, or 6 or more, or 7 or more, or 8 or more, or 9 or more, or 10 or more, or 11 or more, or all 12) residues selected from Arg183, Gly229, Ile230, Gly231, Lys232, Thr233, Ile234, Gly303, Asp305, Glu306, Leu413 and His522.

In another embodiment of the first aspect of the present invention, the binding site further comprises one or more (or 2 or more, or 3 or more, or 4 or more, or 5 or more, or 6 or more, or 7 or more, or 8 or more, or 9 or more, or 10 or more, or 11 or more, or 12 or more, or 13 or more, or 14 or more, or 15 or more, or all 16) residues selected from Gln149, Cys150, Glu152, Asp153, Arg154, Asn155, Ala156, Arg157, Leu158, Glu160, Ser161, Val162, Ser163, Asp302, Trp416 and Tyr565.

A second aspect of the present invention provides a method of inhibiting NLRP3 activation, the method comprising the step of binding a compound to the binding site of the first aspect of the invention. The second aspect of the present invention further provides a compound for use in inhibiting NLRP3 activation, wherein the compound is adapted to bind to the binding site of the first aspect of the invention.

For the purposes of the present invention, where a compound is said to “bind” to a binding site this includes any kind of interaction between the compound and the binding site, including but not limited to covalent binding, non-covalent binding, reversible binding, ionic binding, hydrogen bonding, and Van der Waals bonding.

A third aspect of the present invention provides a method of treating a disease, disorder or condition responsive to NLRP3 inhibition, the method comprising the step of binding a therapeutically effective amount of a compound to the binding site of the first aspect of the invention. The third aspect of the present invention further provides a compound for use in treating a disease, disorder or condition responsive to NLRP3 inhibition, wherein the compound is adapted to bind to the binding site of the first aspect of the invention. The third aspect of the present invention further provides a compound for use in treating a disease, disorder or condition responsive to NLRP3 inhibition, wherein the compound is an antagonist of the binding site of the first aspect of the invention.

In one embodiment of the third aspect of the present invention, the disease, disorder or condition is selected from:
(i) inflammation;
(ii) an auto-immune disease;
(iii) cancer;
(iv) an infection;
(v) a central nervous system disease;
(vi) a metabolic disease;
(vii) a cardiovascular disease;
(viii) a respiratory disease;
(ix) a liver disease;
(x) a renal disease;
(xi) an ocular disease;
(xii) a skin disease;
(xiii) a lymphatic condition;
(xiv) a psychological disorder;
(xv) graft versus host disease;
(xvi) pain;
(xvii) a condition associated with diabetes;
(xviii) a condition associated with arthritis;
(xix) a headache;
(xx) a wound or burn; and
(xxi) any disease where an individual has been determined to carry a germline or somatic non-silent mutation in NLRP3.

In another embodiment of the third aspect of the present invention, the disease, disorder or condition is selected from:
(i) cryopyrin-associated periodic syndromes (CAPS);
(ii) Muckle-Wells syndrome (MWS);
(iii) familial cold autoinflammatory syndrome (FCAS);
(iv) neonatal onset multisystem inflammatory disease (NOMID);
(v) familial Mediterranean fever (FMF);
(vi) pyogenic arthritis, pyoderma gangrenosum and acne syndrome (PAPA);
(vii) hyperimmunoglobulinemia D and periodic fever syndrome (HIDS);
(viii) Tumour Necrosis Factor (TNF) Receptor-Associated Periodic Syndrome (TRAPS);
(ix) systemic juvenile idiopathic arthritis;
(x) adult-onset Still's disease (AOSD);
(xi) relapsing polychondritis;
(xii) Schnitzler's syndrome;
(xiii) Sweet's syndrome;
(xiv) Behcet's disease;
(xv) anti-synthetase syndrome;
(xvi) deficiency of interleukin 1 receptor antagonist (DIRA); and
(xvii) haploinsufficiency of A20 (HA20).

A fourth aspect of the present invention provides a method of reducing cellular or mitochondrial Reactive Oxygen Species (ROS) by inhibiting NLRP3 activation, the method comprising the step of binding a compound to the binding site of the first aspect of the invention. The fourth aspect of the present invention further provides a compound for use in reducing cellular or mitochondrial Reactive Oxygen Species (ROS) by inhibiting NLRP3 activation, wherein the compound is adapted to bind to the binding site of the first aspect of the invention.

In one embodiment of the second, third and fourth aspect of the present invention, the compound is a small molecule (e.g. less than 1,000 Da), peptide, polypeptide, oligonucleotide, protein, antibody or aptamer.

In another embodiment of the second, third and fourth aspect of the present invention, the compound is adapted to bind covalently or non-covalently (i.e. reversibly) to the binding site.

In another embodiment of the second, third and fourth aspect of the present invention, the compound effects inhibition of activation of NLRP3 and thereby prevents ATP displacing ADP from the Walker A and/or Walker B site of NLRP3.

In another embodiment of the second, third and fourth aspect of the present invention, the compound effects inhibition of activation of NLRP3 by binding to one or more residues selected from Arg183, Gly229, Ile230, Gly231, Lys232, Thr233, Ile234, Gly303, Asp305, Glu306, Leu413 and His522. In one embodiment, the compound effects inhibition of activation of NLRP3 by binding to 2 or more (or 3 or more, or 4 or more, or 5 or more, or 6 or more, or 7 or more, or 8 or more, or 9 or more, or 10 or more, or 11 or more, or all 12) residues selected from Arg183, Gly229, Ile230, Gly231, Lys232, Thr233, Ile234, Gly303, Asp305, Glu306, Leu413 and His522. In another embodiment, the compound effects inhibition of activation of NLRP3 by further binding to one or more (or 2 or more, or 3 or more, or 4 or more, or 5 or more, or 6 or more, or 7 or more, or 8 or more, or 9 or more, or 10 or more, or 11 or more, or 12 or more, or 13 or more, or 14 or more, or 15 or more, or all 16) residues selected from Gln149, Cys150, Glu152, Asp153, Arg154, Asn155, Ala156, Arg157, Leu158, Glu160, Ser161, Val162, Ser163, Asp302, Trp416 and Tyr565.

In another embodiment of the second, third and fourth aspect of the present invention, the compound comprises a motif that acts as a phosphonate mimic. For example, the compound may be a sulfoxide, sulfoximine, sulfonyl acetamide, sulfonamide, carbamate, sulfonyl carbamate, urea, sulfonyl urea, or sulfonyl triazole.

A fifth aspect of the present invention provides a method of screening a compound, the method comprising the steps of: (i) exposing the compound to the binding site of the first aspect of the invention, and (ii) determining the extent of binding of the compound to the binding site.

In one embodiment of the fifth aspect of the present invention, the extent of binding of the compound to the binding site is determined by mass spectrometry, NMR (nuclear magnetic resonance), X-ray crystallography, SPR (surface plasmon resonance) or radioligand binding.

In another embodiment of the fifth aspect of the present invention, the method of screening is carried out using a computer. The fifth aspect of the present invention therefore further provides a method of screening a compound, the method comprising the steps of: (i) simulating on a computer exposing the compound to the binding site of the first aspect of the invention, and (ii) determining the extent of binding of the compound to the binding site.

A sixth aspect of the present invention provides a compound identified by a screening method of the fifth aspect of the present invention, or a pharmaceutically acceptable salt, solvate or prodrug thereof.

A seventh aspect of the present invention provides a compound adapted to bind to the binding site of the first aspect of the invention, or a pharmaceutically acceptable salt, solvate or prodrug thereof.

The compounds of the present invention can be used both, in their free base form and their acid addition salt form. For the purposes of this invention, a “salt” of a compound of the present invention includes an acid addition salt. Acid addition salts are preferably pharmaceutically acceptable, non-toxic addition salts with suitable acids, including but not limited to inorganic acids such as hydrohalogenic acids (for example, hydrofluoric, hydrochloric, hydrobromic or hydroiodic acid) or other inorganic acids (for example, nitric, perchloric, sulfuric or phosphoric acid); or organic acids such as organic carboxylic acids (for example, propionic, butyric, glycolic, lactic, mandelic, citric, acetic, benzoic, salicylic, succinic, malic or hydroxysuccinic, tartaric, fumaric, maleic, hydroxymaleic, mucic or galactaric, gluconic, pantothenic or pamoic acid), organic sulfonic acids (for example, methanesulfonic, trifluoromethanesulfonic, ethanesulfonic, 2-hydroxyethanesulfonic, benzenesulfonic, toluene-p-sulfonic, naphthalene-2-sulfonic or camphorsulfonic acid) or amino acids (for example, ornithinic, glutamic or aspartic acid). The acid addition salt may be a mono-, di-, tri- or multi-acid addition salt. A preferred salt is a hydrohalogenic, sulfuric, phosphoric or organic acid addition salt. A preferred salt is a hydrochloric acid addition salt.

Where a compound of the invention includes a quaternary ammonium group, typically the compound is used in its salt form. The counter ion to the quaternary ammonium group may be any pharmaceutically acceptable, non-toxic counter ion. Examples of suitable counter ions include the conjugate bases of the protic acids discussed above in relation to acid addition salts.

The compounds of the present invention can also be used both, in their free acid form and their salt form. For the purposes of this invention, a “salt” of a compound of the present invention includes one formed between a protic acid functionality (such as a carboxylic acid group) of a compound of the present invention and a suitable cation. Suitable cations include, but are not limited to lithium, sodium, potassium, magnesium, calcium and ammonium. The salt may be a mono-, di-, tri- or multi-salt. Preferably the salt is a mono- or di-lithium, sodium, potassium, magnesium, calcium or ammonium salt. More preferably the salt is a mono- or di-sodium salt or a mono- or di-potassium salt.

Preferably any salt is a pharmaceutically acceptable non-toxic salt. However, in addition to pharmaceutically acceptable salts, other salts are included in the present invention, since they have potential to serve as intermediates in the purification or preparation of other, for example, pharmaceutically acceptable salts, or are useful for identification, characterisation or purification of the free acid or base.

The compounds and/or salts of the present invention may be anhydrous or in the form of a hydrate (e.g. a hemihydrate, monohydrate, dihydrate or trihydrate) or other solvate. Such other solvates may be formed with common organic solvents, including but not limited to, alcoholic solvents e.g. methanol, ethanol or isopropanol.

In some embodiments of the present invention, therapeutically inactive prodrugs are provided. Prodrugs are compounds which, when administered to a subject such as a human, are converted in whole or in part to a compound of the invention. In most embodiments, the prodrugs are pharmacologically inert chemical derivatives that can be converted in vivo to the active drug molecules to exert a therapeutic effect. Any of the compounds described herein can be administered as a prodrug to increase the activity, bioavailability, or stability of the compound or to otherwise alter the properties of the compound. Typical examples of prodrugs include compounds that have biologically labile protecting groups on a functional moiety of the active compound. Prodrugs include, but are not limited to, compounds that can be oxidized, reduced, aminated, deaminated, hydroxylated, dehydroxylated, hydrolyzed, dehydrolyzed, alkylated, dealkylated, acylated, deacylated, phosphorylated, and/or dephosphorylated to produce the active compound. The present invention also encompasses salts and solvates of such prodrugs as described above.

The compounds, salts, solvates and prodrugs of the present invention may contain at least one chiral centre. The compounds, salts, solvates and prodrugs may therefore exist in at least two isomeric forms. The present invention encompasses racemic mixtures of the compounds, salts, solvates and prodrugs of the present invention as well as enantiomerically enriched and substantially enantiomerically pure isomers. For the purposes of this invention, a “substantially enantiomerically pure” isomer of a compound comprises less than 5% of other isomers of the same compound, more typically less than 2%, and most typically less than 0.5% by weight.

The compounds, salts, solvates and prodrugs of the present invention may contain any stable isotope including, but not limited to ¹²C, ¹³C, ¹H, ²H (D), ¹⁴N, ¹⁵N, ¹⁶O, ¹⁷O, ¹⁸O, ¹⁹F and ¹²⁷I, and any radioisotope including, but not limited to ¹¹C, ¹⁴C, ³H (T), ¹³N, ¹⁵O, ¹⁸F, ¹²³I, ¹²⁴I, ¹²⁵I and ¹³¹I.

The compounds, salts, solvates and prodrugs of the present invention may be in any polymorphic or amorphous form.

An eighth aspect of the present invention provides a pharmaceutical composition comprising a compound or a pharmaceutically acceptable salt, solvate or prodrug of the sixth or seventh aspect of the present invention, and a pharmaceutically acceptable excipient.

Conventional procedures for the selection and preparation of suitable pharmaceutical formulations are described in, for example, “Aulton's Pharmaceutics—The Design and Manufacture of Medicines”, M. E. Aulton and K. M. G. Taylor, Churchill Livingstone Elsevier, 4^{th} Ed., 2013.

Pharmaceutically acceptable excipients including adjuvants, diluents or carriers that may be used in the pharmaceutical compositions of the invention are those conventionally employed in the field of pharmaceutical formulation, and include, but are not limited to, sugars, sugar alcohols, starches, ion exchangers, alumina, aluminium stearate, lecithin, serum proteins such as human serum albumin, buffer substances such as phosphates, glycerine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinylpyrrolidone, cellulose-based substances, polyethylene glycol, sodium carboxymethylcellulose, polyacrylates, waxes, polyethylene-polyoxypropylene-block polymers, polyethylene glycol and wool fat.

In one embodiment, the pharmaceutical composition of the eighth aspect of the invention additionally comprises one or more further active agents.

In a further embodiment, the pharmaceutical composition of the eighth aspect of the invention may be provided as a part of a kit of parts, wherein the kit of parts comprises the pharmaceutical composition of the eighth aspect of the invention and one or more further pharmaceutical compositions, wherein the one or more further pharmaceutical compositions each comprise a pharmaceutically acceptable excipient and one or more further active agents.

A ninth aspect of the present invention provides a compound or a pharmaceutically acceptable salt, solvate or prodrug of the sixth or seventh aspect of the present invention, or a pharmaceutical composition of the eighth aspect of the present invention, for use in medicine, and/or for use in the treatment or prevention of a disease, disorder or condition. Typically, the use comprises the administration of the compound, salt, solvate, prodrug or pharmaceutical composition to a subject. In one embodiment, the use comprises the co-administration of one or more further active agents.

The term “treatment” as used herein refers equally to curative therapy, and ameliorating or palliative therapy. The term includes obtaining beneficial or desired physiological results, which may or may not be established clinically. Beneficial or desired clinical results include, but are not limited to, the alleviation of symptoms, the prevention of symptoms, the diminishment of extent of disease, the stabilisation (i.e., not worsening) of a condition, the delay or slowing of progression/worsening of a condition/symptom, the amelioration or palliation of a condition/symptom, and remission (whether partial or total), whether detectable or undetectable. The term “palliation”, and variations thereof, as used herein, means that the extent and/or undesirable manifestations of a physiological condition or symptom are lessened and/or time course of the progression is slowed or lengthened, as compared to not administering a compound, salt, solvate, prodrug or pharmaceutical composition of the present invention. The term “prevention” as used herein in relation to a disease, disorder or condition, relates to prophylactic or preventative therapy, as well as therapy to reduce the risk of developing the disease, disorder or condition. The term “prevention” includes both the avoidance of occurrence of the disease, disorder or condition, and the delay in onset of the disease, disorder or condition. Any statistically significant (p≤0.05) avoidance of occurrence, delay in onset or reduction in risk as measured by a controlled clinical trial may be deemed a prevention of the disease, disorder or condition. Subjects amenable to prevention include those at heightened risk of a disease, disorder or condition as identified by genetic or biochemical markers. Typically, the genetic or biochemical markers are appropriate to the disease, disorder or condition under consideration and may include for example, inflammatory biomarkers such as C-reactive protein (CRP) and monocyte chemoattractant protein 1 (MCP-1) in the case of inflammation; total cholesterol, triglycerides, insulin resistance and C-peptide in the case of NAFLD and NASH; and more generally IL-1β and IL-18 in the case of a disease, disorder or condition responsive to NLRP3 inhibition.

A tenth aspect of the invention provides the use of a compound or a pharmaceutically acceptable salt, solvate or prodrug of the sixth or seventh aspect of the present invention, in the manufacture of a medicament for the treatment or prevention of a disease, disorder or condition. Typically, the treatment or prevention comprises the administration of the compound, salt, solvate, prodrug or medicament to a subject. In one embodiment, the treatment or prevention comprises the co-administration of one or more further active agents.

An eleventh aspect of the invention provides a method of treatment or prevention of a disease, disorder or condition, the method comprising the step of administering an effective amount of a compound or a pharmaceutically acceptable salt, solvate or prodrug of the sixth or seventh aspect of the present invention, or a pharmaceutical composition of the eighth aspect of the present invention, to thereby treat or prevent the disease, disorder or condition. In one embodiment, the method further comprises the step of co-administering an effective amount of one or more further active agents. Typically, the administration is to a subject in need thereof.

A twelfth aspect of the invention provides a compound or a pharmaceutically acceptable salt, solvate or prodrug of the sixth or seventh aspect of the present invention, or a pharmaceutical composition of the eighth aspect of the present invention, for use in the treatment or prevention of a disease, disorder or condition in an individual, wherein the individual has a germline or somatic non-silent mutation in NLRP3. The mutation may be, for example, a gain-of-function or other mutation resulting in increased NLRP3 activity. Typically, the use comprises the administration of the compound, salt, solvate, prodrug or pharmaceutical composition to the individual. In one embodiment, the use comprises the co-administration of one or more further active agents. The use may also comprise the diagnosis of an individual having a germline or somatic non-silent mutation in NLRP3, wherein the compound, salt, solvate, prodrug or pharmaceutical composition is administered to an individual on the basis of a positive diagnosis for the mutation. Typically, identification of the mutation in NLRP3 in the individual may be by any suitable genetic or biochemical means.

A thirteenth aspect of the invention provides the use of a compound or a pharmaceutically acceptable salt, solvate or prodrug of the sixth or seventh aspect of the present invention, in the manufacture of a medicament for the treatment or prevention of a disease, disorder or condition in an individual, wherein the individual has a germline or somatic non-silent mutation in NLRP3. The mutation may be, for example, a gain-of-function or other mutation resulting in increased NLRP3 activity. Typically, the treatment or prevention comprises the administration of the compound, salt, solvate, prodrug or medicament to the individual. In one embodiment, the treatment or prevention comprises the co-administration of one or more further active agents. The treatment or prevention may also comprise the diagnosis of an individual having a germline or somatic non-silent mutation in NLRP3, wherein the compound, salt, solvate, prodrug or medicament is administered to an individual on the basis of a positive diagnosis for the mutation. Typically, identification of the mutation in NLRP3 in the individual may be by any suitable genetic or biochemical means.

A fourteenth aspect of the invention provides a method of treatment or prevention of a disease, disorder or condition, the method comprising the steps of diagnosing of an individual having a germline or somatic non-silent mutation in NLRP3, and administering an effective amount of a compound or a pharmaceutically acceptable salt, solvate or prodrug of the sixth or seventh aspect of the present invention, or a pharmaceutical composition of the eighth aspect of the present invention, to the positively diagnosed individual, to thereby treat or prevent the disease, disorder or condition. In one embodiment, the method further comprises the step of co-administering an effective amount of one or more further active agents. Typically, the administration is to a subject in need thereof.

In general embodiments, the disease, disorder or condition may be a disease, disorder or condition of the immune system, the cardiovascular system, the endocrine system, the gastrointestinal tract, the renal system, the hepatic system, the metabolic system, the respiratory system, the central nervous system, may be a cancer or other malignancy, and/or may be caused by or associated with a pathogen.

It will be appreciated that these general embodiments defined according to broad categories of diseases, disorders and conditions are not mutually exclusive. In this regard any particular disease, disorder or condition may be categorized according to more than one of the above general embodiments. A non-limiting example is type I diabetes which is an autoimmune disease and a disease of the endocrine system.

In one embodiment of the ninth to fourteenth aspect of the invention, the disease, disorder or condition is responsive to NLRP3 inhibition. As used herein, the term “NLRP3 inhibition” refers to the complete or partial reduction in the level of activity of NLRP3 and includes, for example, the inhibition of active NLRP3 and/or the inhibition of activation of NLRP3.

There is evidence for a role of NLRP3-induced IL-1 and IL-18 in the inflammatory responses occurring in connection with, or as a result of, a multitude of different disorders (Menu et al., Clinical and Experimental Immunology, 166: 1-15, 2011; Strowig et al., Nature, 481:278-286, 2012).

Genetic diseases in which a role for NLRP3 has been suggested include sickle cell disease (Vogel et al., Blood, 130(Suppl 1): 2234, 2017), and Valosin Containing Protein disease (Nalbandian et al., Inflammation, 40(1): 21-41, 2017).

NLRP3 has been implicated in a number of autoinflammatory diseases, including Familial Mediterranean fever (FMF), TNF receptor associated periodic syndrome (TRAPS), hyperimmunoglobulinemia D and periodic fever syndrome (HIDS), pyogenic arthritis, pyoderma gangrenosum and acne (PAPA), Sweet's syndrome, chronic nonbacterial osteomyelitis (CNO), and acne vulgaris (Cook et al., Eur J Immunol, 40: 595-653, 2010). In particular, NLRP3 mutations have been found to be responsible for a set of rare autoinflammatory diseases known as CAPS (Ozaki et al., J Inflammation Research, 8:15-27, 2015; Schroder et al., Cell, 140: 821-832, 2010; and Menu et al., Clinical and Experimental Immunology, 166: 1-15, 2011). CAPS are heritable diseases characterized by recurrent fever and inflammation and are comprised of three autoinflammatory disorders that form a clinical continuum. These diseases, in order of increasing severity, are familial cold autoinflammatory syndrome (FCAS), Muckle-Wells syndrome (MWS), and chronic infantile cutaneous neurological articular syndrome (CINCA; also called neonatal-onset multisystem inflammatory disease, NOMID), and all have been shown to result from gain-of-function mutations in the NLRP3 gene, which leads to increased secretion of IL-1β.

A number of autoimmune diseases have been shown to involve NLRP3 including, in particular, multiple sclerosis, type 1 diabetes (T1D), psoriasis, rheumatoid arthritis (RA), Behcet's disease, Schnitzler's syndrome, macrophage activation syndrome, Coeliac disease (Masters, Clin Immunol, 147(3): 223-228, 2013; Braddock et al., Nat Rev Drug Disc, 3: 1-10, 2004; Inoue et al., Immunology, 139: 11-18, 2013; Coll et al., Nat Med, 21(3): 248-55, 2015; Scott et al., Clin Exp Rheumatol, 34(1): 88-93, 2016; Pontillo et al., Autoimmunity, 43(8): 583-589, 2010; and Guo et al., Clin Exp Immunol, 194(2): 231-243, 2018), systemic lupus erythematosus (Lu et al., J Immunol, 198(3): 1119-29, 2017) including lupus nephritis (Zhao et al., Arthritis and Rheumatism, 65(12): 3176-3185, 2013), multiple sclerosis (Xu et al., J Cell Biochem, 120(4): 5160-5168, 2019), and systemic sclerosis (Artlett et al., Arthritis Rheum, 63(11): 3563-74, 2011).

NLRP3 has also been shown to play a role in a number of respiratory and lung diseases including chronic obstructive pulmonary disorder (COPD), asthma (including steroid-resistant asthma and eosinophilic asthma), bronchitis, asbestosis, volcanic ash induced inflammation, and silicosis (Cassel et al., Proceedings of the National Academy of Sciences, 105(26): 9035-9040, 2008; Chen et al., ERJ Open Research, 4: 00130-2017, 2018; Chen et al., Toxicological Sciences, 170(2): 462-475, 2019; Damby et al., Front Immun, 8: 2000, 2018; De Nardo et al., Am J Pathol, 184: 42-54, 2014; Lv et al., J Biol Chem, 293(48): 18454, 2018; and Kim et al., Am J Respir Crit Care Med, 196(3): 283-97, 2017).

NLRP3 has also been suggested to have a role in a number of central nervous system conditions, including Parkinson's disease (PD), Alzheimer's disease (AD), dementia, Huntington's disease, cerebral malaria, brain injury from pneumococcal meningitis (Walsh et al., Nature Reviews, 15: 84-97, 2014; Cheng et al., Autophagy, 1-13, 2020; Couturier et al., J Neuroinflamm, 13: 20, 2016; and Dempsey et al., Brain Behav Immun, 61: 306-316, 2017), intracranial aneurysms (Zhang et al., J Stroke & Cerebrovascular Dis, 24(5): 972-979, 2015), intracerebral haemorrhages (ICH) (Ren et al., Stroke, 49(1): 184-192, 2018), cerebral ischemia-reperfusion injuries (Fauzia et al., Front Pharmacol, 9: 1034, 2018; Hong et al., Neural Plasticity, 2018: 8, 2018; Ye et al., Experimental Neurology, 292: 46-55, 2017), general anesthesia neuroinflammation (Fan et al., Front Cell Neurosci, 12: 426, 2018), sepsis-associated encephalopathy (SAE) (Fu et al., Inflammation, 42(1): 306-318, 2019), perioperative neurocognitive disorders including postoperative cognitive dysfunction (POCD) (Fan et al., Front Cell Neurosci, 12: 426, 2018; and Fu et al., International Immunopharmacology, 82: 106317, 2020), early brain injury (subarachnoid haemorrhage SAH) (Luo et al., Brain Res Bull, 146: 320-326, 2019), and traumatic brain injury (Ismael et al., J Neurotrauma, 35(11): 1294-1303, 2018; and Chen et al., Brain Research, 1710: 163-172, 2019).

NRLP3 activity has also been shown to be involved in various metabolic diseases including type 2 diabetes (T2D), atherosclerosis, obesity, gout, pseudo-gout, metabolic syndrome (Wen et al., Nature Immunology, 13: 352-357, 2012; Duewell et al., Nature, 464: 1357-1361, 2010; Strowig et al., Nature, 481: 278-286, 2012), and non-alcoholic steatohepatitis (NASH) (Mridha et al., J Hepatol, 66(5): 1037-46, 2017).

A role for NLRP3 via IL-1β has also been suggested in atherosclerosis (Chen et al., Journal of the American Heart Association, 6(9): e006347, 2017; and Chen et al., Biochem Biophys Res Commun, 495(1): 382-387, 2018), myocardial infarction (van Hout et al., Eur Heart J, 38(11): 828-36, 2017), cardiovascular disease (Janoudi et al., European Heart Journal, 37(25): 1959-1967, 2016), cardiac hypertrophy and fibrosis (Gan et al., Biochim Biophys Acta, 1864(1): 1-10, 2018), heart failure (Sano et al., J Am Coll Cardiol, 71(8): 875-66, 2018), aortic aneurysm and dissection (Wu et al., Arterioscler Thromb Vase Biol, 37(4): 694-706, 2017), cardiac injury induced by metabolic dysfunction (Pavillard et al., Oncotarget, 8(59): 99740-99756, 2017; and Zhang et al., Biochimica et Biophysica Acta, 1863(6): 1556-1567, 2017), atrial fibrillation (Yao et al., Circulation, 138(20): 2227-2242, 2018), hypertension (Gan et al., Biochim Biophys Acta, 1864(1): 1-10, 2018), and other cardiovascular events (Ridker et al., N Engl J Med, doi: 10.1056/NEJM0a1707914, 2017).

Other diseases, disorders and conditions in which NLRP3 has been shown to be involved include:
ocular diseases such as both wet and dry age-related macular degeneration (Doyle et al., Nature Medicine, 18: 791-798, 2012; and Tarallo et al., Cell, 149(4): 847-59, 2012), diabetic retinopathy (Loukovaara et al., Acta Ophthalmol, 95(8): 803-808, 2017) and optic nerve damage (Puyang et al., Sci Rep, 6: 20998, 2016 Feb. 19);
liver diseases including non-alcoholic steatohepatitis (NASH) (Henao-Meija et al., Nature, 482: 179-185, 2012), ischemia reperfusion injury of the liver (Yu et al., Transplantation, 103(2): 353-362, 2019), fulminant hepatitis (Pourcet et al., Gastroenterology, 154(5): 1449-1464, e20, 2018), liver fibrosis (Zhang et al., Parasit Vectors, 12(1): 29, 2019), and liver failure including acute liver failure (Wang et al., Hepatol Res, 48(3): E194-E202, 2018);
kidney diseases including nephrocalcinosis (Anders et al., Kidney Int, 93(3): 656-669, 2018), kidney fibrosis including chronic crystal nephropathy (Ludwig-Portugall et al., Kidney Int, 90(3): 525-39, 2016), obesity related glomerulopathy (Zhao et al., Mediators of Inflammation, article 3172647, 2019), acute kidney injury (Zhang et al., Diabetes, Metabolic Syndrome and Obesity: Targets and Therapy, 12: 1297-1309, 2019), and renal hypertension (Krishnan et al., Br J Pharmacol, 173(4): 752-65, 2016; Krishnan et al., Cardiovasc Res, 115(4): 776-787, 2019; Dinh et al., Aging, 9(6): 1595-1606, 2017);
conditions associated with diabetes including diabetic encephalopathy (Zhai et al., Molecules, 23(3): 522, 2018), diabetic retinopathy (Zhang et al., Cell Death Dis, 8(7): e2941, 2017), diabetic nephropathy (also called diabetic kidney disease) (Chen et al., BMC Complementary and Alternative Medicine, 18: 192, 2018), and diabetic hypoadiponectinemia (Zhang et al., Biochimica et Biophysica Acta (BBA)—Molecular Basis of Disease, 1863(6): 1556-1567, 2017);
inflammatory reactions in the lung and skin (Primiano et al., J Immunol, 197(6): 2421-33, 2016) including lung ischemia-reperfusion injury (Xu et al., Biochemical and Biophysical Research Communications, 503(4): 3031-3037, 2018), epithelial to mesenchymal transition (EMT) (Li et al., Experimental Cell Research, 362(2): 489-497, 2018), contact hypersensitivity (such as bullous pemphigoid (Fang et al., J Dermatol Sci, 83(2): 116-23, 2016)), atopic dermatitis (Niebuhr et al., Allergy, 69(8): 1058-67, 2014), Hidradenitis suppurativa (Alikhan et al., J Am Acad Dermatol, 60(4): 539-61, 2009), acne vulgaris (Qin et al., J Invest Dermatol, 134(2): 381-88, 2014), and sarcoidosis (Jager et al., Am J Respir Crit Care Med, 191: A5816, 2015);
inflammatory reactions in the joints (Braddock et al., Nat Rev Drug Disc, 3: 1-10, 2004) and osteoarthritis (Jin et al., PNAS, 108(36): 14867-14872, 2011);
conditions associated with arthritis including arthritic fever (Verma, Linköping University Medical Dissertations, No. 1250, 2011);
amyotrophic lateral sclerosis (Gugliandolo et al., Inflammation, 41(1): 93-103, 2018);
cystic fibrosis (Iannitti et al., Nat Commun, 7: 10791, 2016);
stroke (Walsh et al., Nature Reviews, 15: 84-97, 2014; Ye et al., Experimental Neurology, 292: 46-55, 2017);
headaches including migraine (He et al., Journal of Neuroinflammation, 16: 78, 2019);
chronic kidney disease (Granata et al., PLoS One, 10(3): e0122272, 2015);
Sjögren's syndrome (Vakrakou et al., Journal of Autoimmunity, 91: 23-33, 2018);
graft-versus-host disease (Takahashi et al., Scientific Reports, 7: 13097, 2017);
sickle cell disease (Vogel et al., Blood, 130(Suppl 1): 2234, 2017); and
colitis and inflammatory bowel diseases including ulcerative colitis and Crohn's disease (Braddock et al., Nat Rev Drug Disc, 3: 1-10, 2004; Neudecker et al., J Exp Med, 214(6): 1737-52, 2017; Wu et al., Mediators Inflamm, 2018: 3048532, 2018; and Lazaridis et al., Dig Dis Sci, 62(9): 2348-56, 2017), and sepsis (intestinal epithelial disruption) (Zhang et al., Dig Dis Sci, 63(1): 81-91, 2018).

Genetic ablation of NLRP3 has been shown to protect from HSD (high sugar diet), HFD (high fat diet) and HSFD-induced obesity (Pavillard et al., Oncotarget, 8(59): 99740-99756, 2017).

The NLRP3 inflammasome has been found to be activated in response to oxidative stress, sunburn (Hasegawa et al., Biochemical and Biophysical Research Communications, 477(3): 329-335, 2016), and UVB irradiation (Schroder et al., Science, 327: 296-300, 2010).

NLRP3 has also been shown to be involved in inflammatory hyperalgesia (Dolunay et al., Inflammation, 40: 366-386, 2017), wound healing (Ito et al., Exp Dermatol, 27(1): 80-86, 2018), burn healing (Chakraborty et al., Exp Dermatol, 27(1): 71-79, 2018), pain including allodynia, multiple sclerosis-associated neuropathic pain (Khan et al., Inflammopharmacology, 26(1): 77-86, 2018), chronic pelvic pain (Zhang et al., Prostate, 79(12): 1439-1449, 2019) and cancer-induced bone pain (Chen et al., Pharmacological Research, 147: 104339, 2019), and intra-amniotic inflammation/infection associated with preterm birth (Faro et al., Biol Reprod, 100(5): 1290-1305, 2019; and Gomez-Lopez et al., Biol Reprod, 100(5): 1306-1318, 2019).

The inflammasome, and NLRP3 specifically, has also been proposed as a target for modulation by various pathogens including bacterial pathogens such as Staphylococcus aureus, including methicillin-resistant Staphylococcus aureus (MRSA) (Cohen et al., Cell Reports, 22(9): 2431-2441, 2018; and Robinson et al., JCI Insight, 3(7): e97470, 2018), Mycobacterium tuberculosis (TB) (Subbarao et al., Scientific Reports, 10: 3709, 2020), Bacillus cereus (Mathur et al., Nat Microbiol, 4: 362-374, 2019), Salmonella typhimurium (Diamond et al., Sci Rep, 7(1): 6861, 2017), and group A streptococcus (LaRock et al., Science Immunology, 1(2): eaah3539, 2016); viruses such as DNA viruses (Amsler et al., Future Virol, 8(4): 357-370, 2013), influenza A virus (Coates et al., Front Immunol, 8: 782, 2017), chikungunya, Ross river virus, and alpha viruses (Chen et al., Nat Microbiol, 2(10): 1435-1445, 2017); fungal pathogens such as Candida albicans (Tucey et al., mSphere, 1(3), pii: e00074-16, 2016); and other pathogens such as T. gondii (Gov et al., J Immunol, 199(8): 2855-2864, 2017), helminth worms (Alhallaf et al., Cell Reports, 23(4): 1085-1098, 2018), leishmania (Novais et al., PLoS Pathogens, 13(2): e1006196, 2017), and plasmodium (Strangward et al., PNAS, 115(28): 7404-7409, 2018). NLRP3 has been shown to be required for the efficient control of viral, bacterial, fungal, and helminth pathogen infections (Strowig et al., Nature, 481: 278-286, 2012). NLRP3 activity has also been associated with increased susceptibility to viral infection such as by the human immunodeficiency virus (HIV) (Pontillo et al., J Aquir Immune Defic Syndr, 54(3): 236-240, 2010). An increased risk for early mortality amongst patients co-infected with HIV and Mycobacterium tuberculosis (TB) has also been associated with NLRP3 activity (Ravimohan et al., Open Forum Infectious Diseases, 5(5): ofy075, 2018).

NLRP3 has been implicated in the pathogenesis of many cancers (Menu et al., Clinical and Experimental Immunology, 166: 1-15, 2011; and Masters, Clin Immunol, 147(3): 223-228, 2013). For example, several previous studies have suggested a role for IL-1β in cancer invasiveness, growth and metastasis, and inhibition of IL-1β with canakinumab has been shown to reduce the incidence of lung cancer and total cancer mortality in a randomised, double-blind, placebo-controlled trial (Ridker et al., Lancet, S0140-6736(17)32247-X, 2017). Inhibition of the NLRP3 inflammasome or IL-1β has also been shown to inhibit the proliferation and migration of lung cancer cells in vitro (Wang et al., Oncol Rep, 35(4): 2053-64, 2016), and NLRP3 has been shown to suppress NK cell-mediated control of carcinogenesis and metastases (Chow et al., Cancer Res, 72(22): 5721-32, 2012). A role for the NLRP3 inflammasome has been suggested in myelodysplastic syndromes (Basiorka et al., Blood, 128(25): 2960-2975, 2016) and also in the carcinogenesis of various other cancers including glioma (Li et al., Am J Cancer Res, 5(1): 442-449, 2015), colon cancer (Allen et al., J Exp Med, 207(5): 1045-56, 2010), melanoma (Dunn et al., Cancer Lett, 314(1): 24-33, 2012), breast cancer (Guo et al., Scientific Reports, 6: 36107, 2016), inflammation-induced tumours (Allen et al., J Exp Med, 207(5): 1045-56, 2010; and Hu et al., PNAS, 107(50): 21635-40, 2010), multiple myeloma (Li et al., Hematology, 21(3): 144-51, 2016), and squamous cell carcinoma of the head and neck (Huang et al., J Exp Clin Cancer Res, 36(1): 116, 2017; and Chen et al., Cellular and Molecular Life Sciences, 75: 2045-2058, 2018). Activation of the NLRP3 inflammasome has also been shown to mediate chemoresistance of tumour cells to 5-fluorouracil (Feng et al., J Exp Clin Cancer Res, 36(1): 81, 2017), and activation of the NLRP3 inflammasome in peripheral nerves contributes to chemotherapy-induced neuropathic pain (Jia et al., Mol Pain, 13: 1-11, 2017).

Accordingly, any of the diseases, disorders or conditions listed above may be treated or prevented in accordance with the ninth to fourteenth aspect of the present invention. Particular examples of diseases, disorders or conditions which may be responsive to NLRP3 inhibition and which may be treated or prevented in accordance with the ninth to fourteenth aspect of the present invention include:

(i) inflammation, including inflammation occurring as a result of an inflammatory disorder, e.g. an autoinflammatory disease, inflammation occurring as a symptom of a non-inflammatory disorder, inflammation occurring as a result of infection, or inflammation secondary to trauma, injury or autoimmunity;

(ii) auto-immune diseases such as acute disseminated encephalitis, Addison's disease, ankylosing spondylitis, antiphospholipid antibody syndrome (APS), anti-synthetase syndrome, aplastic anemia, autoimmune adrenalitis, autoimmune hepatitis, autoimmune oophoritis, autoimmune polyglandular failure, autoimmune thyroiditis, Coeliac disease including paediatric Coeliac disease, Crohn's disease, type 1 diabetes (T1D), Goodpasture's syndrome, Graves' disease, Guillain-Barre syndrome (GBS), Hashimoto's disease, idiopathic thrombocytopenic purpura, Kawasaki's disease, lupus erythematosus including systemic lupus erythematosus (SLE), multiple sclerosis (MS) including primary progressive multiple sclerosis (PPMS), secondary progressive multiple sclerosis (SPMS) and relapsing remitting multiple sclerosis (RRMS), myasthenia gravis, opsoclonus myoclonus syndrome (OMS), optic neuritis, Ord's thyroiditis, pemphigus, pernicious anaemia, polyarthritis, primary biliary cirrhosis, rheumatoid arthritis (RA), psoriatic arthritis, juvenile idiopathic arthritis or Still's disease, refractory gouty arthritis, Reiter's syndrome, Sjögren's syndrome, systemic sclerosis, a systemic connective tissue disorder, Takayasu's arteritis, temporal arteritis, warm autoimmune hemolytic anemia, Wegener's granulomatosis, alopecia universalis, Behcet's disease, Chagas' disease, dysautonomia, endometriosis, hidradenitis suppurativa (HS), interstitial cystitis, neuromyotonia, psoriasis, sarcoidosis, scleroderma, ulcerative colitis, Schnitzler's syndrome, macrophage activation syndrome, Blau syndrome, vitiligo or vulvodynia;

(iii) cancer including lung cancer, pancreatic cancer, gastric cancer, myelodysplastic syndrome, leukaemia including acute lymphocytic leukaemia (ALL) and acute myeloid leukaemia (AML), adrenal cancer, anal cancer, basal and squamous cell skin cancer, squamous cell carcinoma of the head and neck, bile duct cancer, bladder cancer, bone cancer, brain and spinal cord tumours, breast cancer, cervical cancer, chronic lymphocytic leukaemia (CLL), chronic myeloid leukaemia (CML), chronic myelomonocytic leukaemia (CMML), colorectal cancer, endometrial cancer, oesophagus cancer, Ewing family of tumours, eye cancer, gallbladder cancer, gastrointestinal carcinoid tumours, gastrointestinal stromal tumour (GIST), gestational trophoblastic disease, glioma, Hodgkin lymphoma, Kaposi sarcoma, kidney cancer, laryngeal and hypopharyngeal cancer, liver cancer, lung carcinoid tumour, lymphoma including cutaneous T cell lymphoma, malignant mesothelioma, melanoma skin cancer, Merkel cell skin cancer, multiple myeloma, nasal cavity and paranasal sinuses cancer, nasopharyngeal cancer, neuroblastoma, non-Hodgkin lymphoma, non-small cell lung cancer, oral cavity and oropharyngeal cancer, osteosarcoma, ovarian cancer, penile cancer, pituitary tumours, prostate cancer, retinoblastoma, rhabdomyosarcoma, salivary gland cancer, skin cancer, small cell lung cancer, small intestine cancer, soft tissue sarcoma, stomach cancer, testicular cancer, thymus cancer, thyroid cancer including anaplastic thyroid cancer, uterine sarcoma, vaginal cancer, vulvar cancer, Waldenstrom macroglobulinemia, and Wilms tumour;

(iv) infections including viral infections (e.g. from influenza virus, human immunodeficiency virus (HIV), alphavirus (such as Chikungunya and Ross River virus), flaviviruses (such as Dengue virus and Zika virus), herpes viruses (such as Epstein Barr virus, cytomegalovirus, Varicella-zoster virus, and KSHV), poxviruses (such as vaccinia virus (Modified vaccinia virus Ankara) and Myxoma virus), adenoviruses (such as Adenovirus 5), or papillomavirus), bacterial infections (e.g. from Staphylococcus aureus (including MRSA), Helicobacter pylori, Bacillus anthracis, Bacillus cereus, Bordatella pertussis, Burkholderia pseudomallei, Corynebacterium diptheriae, Clostridium tetani, Clostridium botulinum, Streptococcus pneumoniae, Streptococcus pyogenes, Listeria monocytogenes, Hemophilus influenzae, Pasteurella multicida, Shigella dysenteriae, Mycobacterium tuberculosis, Mycobacterium leprae, Mycoplasma pneumoniae, Mycoplasma hominis, Neisseria meningitidis, Neisseria gonorrhoeae, Rickettsia rickettsii, Legionella pneumophila, Klebsiella pneumoniae, Pseudomonas aeruginosa, Propionibacterium acnes, Treponema pallidum, Chlamydia trachomatis, Vibrio cholerae, Salmonella typhimurium, Salmonella typhi, Borrelia burgdorferi, Uropathogenic Escherichia coli (UPEC) or Yersinia pestis), fungal infections (e.g. from Candida or Aspergillus species), protozoan infections (e.g. from Plasmodium, Babesia, Giardia, Entamoeba, Leishmania or Trypanosomes), helminth infections (e.g. from schistosoma, roundworms, tapeworms or flukes), prion infections, and co-infections with any of the aforementioned (e.g. with HIV and Mycobacterium tuberculosis);

(v) central nervous system diseases such as Parkinson's disease, Alzheimer's disease, dementia, motor neuron disease, Huntington's disease, cerebral malaria, brain injury from pneumococcal meningitis, intracranial aneurysms, intracerebral haemorrhages, sepsis-associated encephalopathy, perioperative neurocognitive disorder, postoperative cognitive dysfunction, early brain injury, traumatic brain injury, cerebral ischemia-reperfusion injury, stroke, general anesthesia neuroinflammation and amyotrophic lateral sclerosis;

(vi) metabolic diseases such as type 2 diabetes (T2D), atherosclerosis, obesity, gout, and pseudo-gout;

(vii) cardiovascular diseases such as hypertension, ischaemia, reperfusion injury including post-MI ischemic reperfusion injury, stroke including ischemic stroke, transient ischemic attack, myocardial infarction including recurrent myocardial infarction, heart failure including congestive heart failure and heart failure with preserved ejection fraction, cardiac hypertrophy and fibrosis, embolism, aneurysms including abdominal aortic aneurysm, metabolism induced cardiac injury, and pericarditis including Dressler's syndrome;

(viii) respiratory diseases including chronic obstructive pulmonary disorder (COPD), asthma such as allergic asthma, eosinophilic asthma, and steroid-resistant asthma, asbestosis, silicosis, volcanic ash induced inflammation, nanoparticle induced inflammation, cystic fibrosis and idiopathic pulmonary fibrosis;

(ix) liver diseases including non-alcoholic fatty liver disease (NAFLD) and non-alcoholic steatohepatitis (NASH) including advanced fibrosis stages F3 and F4, alcoholic fatty liver disease (AFLD), alcoholic steatohepatitis (ASH), ischemia reperfusion injury of the liver, fulminant hepatitis, liver fibrosis, and liver failure including acute liver failure;

(x) renal diseases including chronic kidney disease, oxalate nephropathy, nephrocalcinosis, glomerulonephritis, diabetic nephropathy, obesity related glomerulopathy, kidney fibrosis including chronic crystal nephropathy, acute renal failure, acute kidney injury, and renal hypertension;

(xi) ocular diseases including those of the ocular epithelium, age-related macular degeneration (AMD) (dry and wet), Sjögren's syndrome, uveitis, corneal infection, diabetic retinopathy, optic nerve damage, dry eye, and glaucoma;

(xii) skin diseases including dermatitis such as contact dermatitis and atopic dermatitis, contact hypersensitivity, psoriasis, sunburn, skin lesions, hidradenitis suppurativa (HS), other cyst-causing skin diseases, pyoderma gangrenosum, and acne vulgaris including acne conglobata;

(xiii) lymphatic conditions such as lymphangitis and Castleman's disease;

(xiv) psychological disorders such as depression and psychological stress;

(xv) graft versus host disease;

(xvi) pain such as pelvic pain, hyperalgesia, allodynia including mechanical allodynia, neuropathic pain including multiple sclerosis-associated neuropathic pain, and cancer-induced bone pain;

(xvii) conditions associated with diabetes including diabetic encephalopathy, diabetic retinopathy, diabetic nephropathy, diabetic vascular endothelial dysfunction, and diabetic hypoadiponectinemia;

(xviii) conditions associated with arthritis including arthritic fever;

(xix) headache including cluster headaches, idiopathic intracranial hypertension, migraine, low pressure headaches (e.g. post-lumbar puncture), Short-Lasting Unilateral Neuralgiform Headache With Conjunctival Injection and Tearing (SUNCT), and tension-type headaches;

(xx) wounds and burns, including skin wounds and skin burns; and

(xxi) any disease where an individual has been determined to carry a germline or somatic non-silent mutation in NLRP3.

In one embodiment, the disease, disorder or condition is selected from:

(i) cancer;

(ii) an infection;

(iii) a central nervous system disease;

(iv) a cardiovascular disease;

(v) a liver disease;

(vi) an ocular disease; or

(vii) a skin disease.

More typically, the disease, disorder or condition is selected from:

(i) cancer;

(ii) an infection;

(iii) a central nervous system disease; or

(iv) a cardiovascular disease.

In one embodiment, the disease, disorder or condition is selected from:

(i) acne conglobata;

(ii) atopic dermatitis;

(iii) Alzheimer's disease;

(iv) amyotrophic lateral sclerosis;

(v) age-related macular degeneration (AMD);

(vi) anaplastic thyroid cancer;

(vii) cryopyrin-associated periodic syndromes (CAPS);

(viii) contact dermatitis;

(ix) cystic fibrosis;

(x) congestive heart failure;

(xi) chronic kidney disease;

(xii) Crohn's disease;

(xiii) familial cold autoinflammatory syndrome (FCAS);

(xiv) Huntington's disease;

(xv) heart failure;

(xvi) heart failure with preserved ejection fraction;

(xvii) ischemic reperfusion injury;

(xviii) juvenile idiopathic arthritis;

(xix) myocardial infarction;

(xx) macrophage activation syndrome;

(xxi) myelodysplastic syndrome;

(xxii) multiple myeloma;

(xxiii) motor neuron disease;

(xxiv) multiple sclerosis;

(xxv) Muckle-Wells syndrome;

(xxvi) non-alcoholic steatohepatitis (NASH);

(xxvii) neonatal-onset multisystem inflammatory disease (NOMID);

(xxviii) Parkinson's disease;

(xxix) sickle cell disease;

(xxx) systemic juvenile idiopathic arthritis;

(xxxi) systemic lupus erythematosus;

(xxxii) traumatic brain injury;

(xxxiii) transient ischemic attack;

(xxxiv) ulcerative colitis; or

(xxxv) Valosin Containing Protein disease.

In another embodiment of the ninth to fourteenth aspect of the present invention, the treatment or prevention comprises a reduction in susceptibility to viral infection. For instance, the treatment or prevention may comprise a reduction in susceptibility to HIV infection.

In a further typical embodiment of the invention, the disease, disorder or condition is inflammation. Examples of inflammation that may be treated or prevented in accordance with the ninth to fourteenth aspect of the present invention include inflammatory responses occurring in connection with, or as a result of:

(i) a skin condition such as contact hypersensitivity, bullous pemphigoid, sunburn, psoriasis, atopical dermatitis, contact dermatitis, allergic contact dermatitis, seborrhoetic dermatitis, lichen planus, scleroderma, pemphigus, epidermolysis bullosa, urticaria, erythemas, or alopecia;

(ii) a joint condition such as osteoarthritis, systemic juvenile idiopathic arthritis, adult-onset Still's disease, relapsing polychondritis, rheumatoid arthritis, juvenile chronic arthritis, gout, or a seronegative spondyloarthropathy (e.g. ankylosing spondylitis, psoriatic arthritis or Reiter's disease);

(iii) a muscular condition such as polymyositis or myasthenia gravis;

(iv) a gastrointestinal tract condition such as inflammatory bowel disease (including Crohn's disease and ulcerative colitis), colitis, gastric ulcer, Coeliac disease, proctitis, pancreatitis, eosinopilic gastro-enteritis, mastocytosis, antiphospholipid syndrome, or a food-related allergy which may have effects remote from the gut (e.g., migraine, rhinitis or eczema);

(v) a respiratory system condition such as chronic obstructive pulmonary disease (COPD), asthma (including eosinophilic, bronchial, allergic, intrinsic, extrinsic or dust asthma, and particularly chronic or inveterate asthma, such as late asthma and airways hyper-responsiveness), bronchitis, rhinitis (including acute rhinitis, allergic rhinitis, atrophic rhinitis, chronic rhinitis, rhinitis caseosa, hypertrophic rhinitis, rhinitis pumlenta, rhinitis sicca, rhinitis medicamentosa, membranous rhinitis, seasonal rhinitis e.g. hay fever, and vasomotor rhinitis), sinusitis, idiopathic pulmonary fibrosis (IPF), sarcoidosis, farmer's lung, silicosis, asbestosis, volcanic ash induced inflammation, adult respiratory distress syndrome, hypersensitivity pneumonitis, or idiopathic interstitial pneumonia;

(vi) a vascular condition such as atherosclerosis, Behcet's disease, vasculitides, or Wegener's granulomatosis;

(vii) an autoimmune condition such as systemic lupus erythematosus, Sjögren's syndrome, systemic sclerosis, Hashimoto's thyroiditis, type I diabetes, idiopathic thrombocytopenia purpura, or Graves disease;

(viii) an ocular condition such as uveitis, allergic conjunctivitis, or vernal conjunctivitis;

(ix) a nervous condition such as multiple sclerosis or encephalomyelitis;

(x) an infection or infection-related condition, such as Acquired Immunodeficiency Syndrome (AIDS), acute or chronic bacterial infection, acute or chronic parasitic infection, acute or chronic viral infection, acute or chronic fungal infection, meningitis, hepatitis (A, B or C, or other viral hepatitis), peritonitis, pneumonia, epiglottitis, malaria, dengue hemorrhagic fever, leishmaniasis, streptococcal myositis, Mycobacterium tuberculosis (including Mycobacterium tuberculosis and HIV co-infection), Mycobacterium avium intracellulare, Pneumocystis carinii pneumonia, orchitis/epidydimitis, legionella, Lyme disease, influenza A, Epstein-Barr virus infection, viral encephalitis/aseptic meningitis, or pelvic inflammatory disease;

(xi) a renal condition such as mesangial proliferative glomerulonephritis, nephrotic syndrome, nephritis, glomerular nephritis, obesity related glomerulopathy, acute renal failure, acute kidney injury, uremia, nephritic syndrome, kidney fibrosis including chronic crystal nephropathy, or renal hypertension;

(xii) a lymphatic condition such as Castleman's disease;

(xiii) a condition of, or involving, the immune system, such as hyper IgE syndrome, lepromatous leprosy, familial hemophagocytic lymphohistiocytosis, or graft versus host disease;

(xiv) a hepatic condition such as chronic active hepatitis, non-alcoholic steatohepatitis (NASH), alcohol-induced hepatitis, non-alcoholic fatty liver disease (NAFLD), alcoholic fatty liver disease (AFLD), alcoholic steatohepatitis (ASH), primary biliary cirrhosis, fulminant hepatitis, liver fibrosis, or liver failure;

(xv) a cancer, including those cancers listed above;

(xvi) a burn, wound, trauma, haemorrhage or stroke;

(xvii) radiation exposure;

(xviii) a metabolic disease such as type 2 diabetes (T2D), atherosclerosis, obesity, gout or pseudo-gout; and/or

(xix) pain such as inflammatory hyperalgesia, pelvic pain, allodynia, neuropathic pain, or cancer-induced bone pain.

In one embodiment of the ninth to fourteenth aspect of the present invention, the disease, disorder or condition is an autoinflammatory disease such as cryopyrin-associated periodic syndromes (CAPS), Muckle-Wells syndrome (MWS), familial cold autoinflammatory syndrome (FCAS), familial Mediterranean fever (FMF), neonatal onset multisystem inflammatory disease (NOMID), Tumour Necrosis Factor (TNF) Receptor-Associated Periodic Syndrome (TRAPS), hyperimmunoglobulinemia D and periodic fever syndrome (HIDS), deficiency of interleukin 1 receptor antagonist (DIRA), Majeed syndrome, pyogenic arthritis, pyoderma gangrenosum and acne syndrome (PAPA), adult-onset Still's disease (AOSD), haploinsufficiency of A20 (HA20), pediatric granulomatous arthritis (PGA), PLCG2-associated antibody deficiency and immune dysregulation (PLAID), PLCG2-associated autoinflammatory, antibody deficiency and immune dysregulation (APLAID), or sideroblastic anaemia with B-cell immunodeficiency, periodic fevers and developmental delay (SIFD).

Examples of diseases, disorders or conditions which may be responsive to NLRP3 inhibition and which may be treated or prevented in accordance with the ninth to fourteenth aspect of the present invention are listed above. Some of these diseases, disorders or conditions are substantially or entirely mediated by NLRP3 inflammasome activity, and NLRP3-induced IL-1β and/or IL-18. As a result, such diseases, disorders or conditions may be particularly responsive to NLRP3 inhibition and may be particularly suitable for treatment or prevention in accordance with the ninth to fourteenth aspect of the present invention. Examples of such diseases, disorders or conditions include cryopyrin-associated periodic syndromes (CAPS), Muckle-Wells syndrome (MWS), familial cold autoinflammatory syndrome (FCAS), neonatal onset multisystem inflammatory disease (NOMID), familial Mediterranean fever (FMF), pyogenic arthritis, pyoderma gangrenosum and acne syndrome (PAPA), hyperimmunoglobulinemia D and periodic fever syndrome (HIDS), Tumour Necrosis Factor (TNF) Receptor-Associated Periodic Syndrome (TRAPS), systemic juvenile idiopathic arthritis, adult-onset Still's disease (AOSD), relapsing polychondritis, Schnitzler's syndrome, Sweet's syndrome, Behcet's disease, anti-synthetase syndrome, deficiency of interleukin 1 receptor antagonist (DIRA), and haploinsufficiency of A20 (HA20).

Moreover, some of the diseases, disorders or conditions mentioned above arise due to mutations in NLRP3, in particular, resulting in increased NLRP3 activity. As a result, such diseases, disorders or conditions may be particularly responsive to NLRP3 inhibition and may be particularly suitable for treatment or prevention in accordance with the ninth to fourteenth aspect of the present invention. Examples of such diseases, disorders or conditions include cryopyrin-associated periodic syndromes (CAPS), Muckle-Wells syndrome (MWS), familial cold autoinflammatory syndrome (FCAS), and neonatal onset multisystem inflammatory disease (NOMID).

A fifteenth aspect of the present invention provides a method of inhibiting NLRP3 activation, the method comprising the use of a compound or a pharmaceutically acceptable salt, solvate or prodrug of the sixth or seventh aspect of the present invention, or a pharmaceutical composition of the eighth aspect of the present invention, to inhibit NLRP3 activation.

In one embodiment of the fifteenth aspect of the present invention, the method comprises the use of a compound or a pharmaceutically acceptable salt, solvate or prodrug of the sixth or seventh aspect of the present invention, or a pharmaceutical composition of the eighth aspect of the present invention, in combination with one or more further active agents.

In one embodiment of the fifteenth aspect of the present invention, the method is performed ex vivo or in vitro, for example in order to analyse the effect on cells of NLRP3 inhibition.

In another embodiment of the fifteenth aspect of the present invention, the method is performed in vivo. For example, the method may comprise the step of administering an effective amount of a compound or a pharmaceutically acceptable salt, solvate or prodrug of the sixth or seventh aspect of the present invention, or a pharmaceutical composition of the eighth aspect of the present invention, to thereby inhibit NLRP3. In one embodiment, the method further comprises the step of co-administering an effective amount of one or more further active agents. Typically, the administration is to a subject in need thereof.

Alternately, the method of the fifteenth aspect of the invention may be a method of inhibiting NLRP3 in a non-human animal subject, the method comprising the steps of administering the compound, salt, solvate, prodrug or pharmaceutical composition to the non-human animal subject and optionally subsequently mutilating or sacrificing the non-human animal subject. Typically, such a method further comprises the step of analysing one or more tissue or fluid samples from the optionally mutilated or sacrificed non-human animal subject. In one embodiment, the method further comprises the step of co-administering an effective amount of one or more further active agents.

A sixteenth aspect of the invention provides a compound or a pharmaceutically acceptable salt, solvate or prodrug of the sixth or seventh aspect of the present invention, or a pharmaceutical composition of the eighth aspect of the present invention, for use in the inhibition of NLRP3. Typically, the use comprises the administration of the compound, salt, solvate, prodrug or pharmaceutical composition to a subject. In one embodiment, the compound, salt, solvate, prodrug or pharmaceutical composition is co-administered with one or more further active agents.

A seventeenth aspect of the invention provides the use of a compound or a pharmaceutically acceptable salt, solvate or prodrug of the sixth or seventh aspect of the present invention, in the manufacture of a medicament for the inhibition of NLRP3. Typically, the inhibition comprises the administration of the compound, salt, solvate, prodrug or medicament to a subject. In one embodiment, the compound, salt, solvate, prodrug or medicament is co-administered with one or more further active agents.

In any embodiment of any of the ninth to seventeenth aspects of the present invention that comprises the use or co-administration of one or more further active agents, the one or more further active agents may comprise for example one, two or three different further active agents.

The one or more further active agents may be used or administered prior to, simultaneously with, sequentially with or subsequent to each other and/or to the compound or the pharmaceutically acceptable salt, solvate or prodrug of the sixth or seventh aspect of the present invention, or the pharmaceutical composition of the eighth aspect of the present invention. Where the one or more further active agents are administered simultaneously with the compound or the pharmaceutically acceptable salt, solvate or prodrug of the sixth or seventh aspect of the present invention, a pharmaceutical composition of the eighth aspect of the present invention may be administered wherein the pharmaceutical composition additionally comprises the one or more further active agents.

In one embodiment of any of the ninth to seventeenth aspects of the present invention that comprises the use or co-administration of one or more further active agents, the one or more further active agents are selected from:

(i) chemotherapeutic agents;

(ii) antibodies;

(iii) alkylating agents;

(iv) anti-metabolites;

(v) anti-angiogenic agents;

(vi) plant alkaloids and/or terpenoids;

(vii) topoisomerase inhibitors;

(viii) mTOR inhibitors;

(ix) stilbenoids;

(x) STING agonists;

(xi) cancer vaccines;

(xii) immunomodulatory agents;

(xiii) antibiotics;

(xiv) anti-fungal agents;

(xv) anti-helminthic agents; and/or

(xvi) other active agents.

It will be appreciated that these general embodiments defined according to broad categories of active agents are not mutually exclusive. In this regard any particular active agent may be categorized according to more than one of the above general embodiments. A non-limiting example is urelumab which is an antibody that is an immunomodulatory agent for the treatment of cancer.

As will be understood, where the further active agent is a small chemical entity, any reference to a specific small chemical entity below is to be understood to encompass all salt, hydrate, solvate, polymorphic and prodrug forms of the specific small chemical entity. Similarly, where the further active agent is a biologic such as a monoclonal antibody, any reference to a specific biologic below is to be understood to encompass all biosimilars thereof.

In some embodiments, the one or more chemotherapeutic agents are selected from abiraterone acetate, altretamine, amsacrine, anhydrovinblastine, auristatin, azacitidine, 5-azacytidine, azathioprine, adriamycin, bexarotene, bicalutamide, BMS 184476, bleomycin, bortezomib, N,N-dimethyl-L-valyl-L-valyl-N-methyl-L-valyl-L-prolyl-L-proline-t-butylamide, cisplatin, carboplatin, carboplatin cyclophosphamide, chlorambucil, cachectin, cemadotin, cyclophosphamide, carmustine, cladribine, cryptophycin, cytarabine, docetaxel, doxetaxel, doxorubicin, dacarbazine (DTIC), dactinomycin, daunorubicin, decitabine, dolastatin, etoposide, etoposide phosphate, enzalutamide (MDV3100), 5-fluorouracil, fludarabine, flutamide, gemcitabine, hydroxyurea and hydroxyureataxanes, idarubicin, ifosfamide, irinotecan, ixazomib, lenalidomide, lenalidomide-dexamethasone, leucovorin, lonidamine, lomustine (CCNU), larotaxel (RPR109881), mechlorethamine, mercaptopurine, methotrexate, mitomycin C, mitoxantrone, melphalan, mivobulin, 3′,4′-didehydro-4′-deoxy-8′-norvin-caleukoblastine, nilutamide, oxaliplatin, onapristone, prednimustine, procarbazine, paclitaxel, platinum-containing anti-cancer agents, 2,3,4,5,6-pentafluoro-N-(3-fluoro-4-methoxyphenyl)benzene sulfonamide, prednimustine, procarbazine, revlimid, rhizoxin, sertenef, streptozocin, stramustine phosphate, tretinoin, tasonermin, taxol, topotecan, tamoxifen, teniposide, taxane, tegafur/uracil, thalidomide, vincristine, vinblastine, vinorelbine, vindesine, vindesine sulfate, and/or vinflunine.

Alternatively or in addition, the one or more chemotherapeutic agents may be selected from CD59 complement fragment, fibronectin fragment, gro-beta (CXCL2), heparinases, heparin hexasaccharide fragment, human chorionic gonadotropin (hCG), Type I interferon ligands such as interferon alpha and interferon beta, Type I interferon mimetics, Type II interferon ligands such as interferon gamma, Type II interferon mimetics, interferon inducible protein (IP-10), kringle 5 (plasminogen fragment), metalloproteinase inhibitors (TIMPs), 2-methoxyestradiol, placental ribonuclease inhibitor, plasminogen activator inhibitor, platelet factor-4 (PF4), prolactin 16 kD fragment, proliferin-related protein (PRP), various retinoids, tetrahydrocortisol-S, thrombospondin-1 (TSP-1), transforming growth factor-beta (TGF-β), vasculostatin, vasostatin (calreticulin fragment), cytokines (including interleukins, such as interleukin-1, interleukin-2, interleukin-5, interleukin-10, interleukin-12, and interleukin-33), interleukin-1 ligands and mimetics (such as rilonacept, anakinra, and anakinra-dexamethasone), interleukin-2 ligands and mimetics, interleukin-5 ligands and mimetics, interleukin-10 ligands and mimetics, interleukin-12 ligands and mimetics, and/or interleukin-33 ligands and mimetics.

In some embodiments, the one or more antibodies may comprise one or more monoclonal antibodies. In some embodiments, the one or more antibodies are anti-TNFα and/or anti-IL-6 antibodies, in particular anti-TNFα and/or anti-IL-6 monoclonal antibodies. In some embodiments, the one or more antibodies are selected from abatacept, abciximab, adalimumab, alemtuzumab, atezolizumab, atlizumab, avelumab, basiliximab, belimumab, benralizumab, bevacizumab, bretuximab vedotin, brodalumab, canakinumab, cetuximab, ceertolizumab pegol, daclizumab, denosumab, dupilumab, durvalumab, eculizumab, efalizumab, elotuzumab, gemtuzumab, golimumab, guselkumab, ibritumomab tiuxetan, infliximab, ipilimumab, ixekizumab, mepolizumab, muromonab-CD3, natalizumab, nivolumab, ofatumumab, omalizumab, palivizumab, panitumuab, pembrolizumab, ranibizumab, reslizumab, risankizumab, rituximab, sarilumab, secukinumab, siltuximab, tildrakizumab, tocilizumab, tositumomab, trastuzumab, and/or ustekinumab.

In some embodiments, the one or more alkylating agents may comprise an agent capable of alkylating nucleophilic functional groups under conditions present in cells, including, for example, cancer cells. In some embodiments, the one or more alkylating agents are selected from cisplatin, carboplatin, mechlorethamine, cyclophosphamide, chlorambucil, ifosfamide and/or oxaliplatin. In some embodiments, the alkylating agent may function by impairing cell function by forming covalent bonds with amino, carboxyl, sulfhydryl, and/or phosphate groups in biologically important molecules. In some embodiments, the alkylating agent may function by modifying a cell's DNA.

In some embodiments, the one or more anti-metabolites may comprise an agent capable of affecting or preventing RNA or DNA synthesis. In some embodiments, the one or more anti-metabolites are selected from azathioprine and/or mercaptopurine.

In some embodiments, the one or more anti-angiogenic agents are selected from thalidomide, lenalidomide, endostatin, angiogenin inhibitors, angioarrestin, angiostatin (plasminogen fragment), basement-membrane collagen-derived anti-angiogenic factors (tumstatin, canstatin, or arrestin), anti-angiogenic antithrombin III, and/or cartilage-derived inhibitor (CDI).

In some embodiments, the one or more plant alkaloids and/or terpenoids may prevent microtubule function. In some embodiments, the one or more plant alkaloids and/or terpenoids are selected from a vinca alkaloid, a podophyllotoxin and/or a taxane. In some embodiments, the one or more vinca alkaloids may be derived from the Madagascar periwinkle, Catharanthus roseus (formerly known as Vinca rosea), and may be selected from vincristine, vinblastine, vinorelbine and/or vindesine. In some embodiments, the one or more taxanes are selected from taxol, paclitaxel, docetaxel and/or ortataxel. In some embodiments, the one or more podophyllotoxins are selected from an etoposide and/or teniposide.

In some embodiments, the one or more topoisomerase inhibitors are selected from a type I topoisomerase inhibitor and/or a type II topoisomerase inhibitor, and may interfere with transcription and/or replication of DNA by interfering with DNA supercoiling. In some embodiments, the one or more type I topoisomerase inhibitors may comprise a camptothecin, which may be selected from exatecan, irinotecan, lurtotecan, topotecan, BNP 1350, CKD 602, DB 67 (AR67) and/or ST 1481. In some embodiments, the one or more type II topoisomerase inhibitors may comprise an epipodophyllotoxin, which may be selected from an amsacrine, etoposid, etoposide phosphate and/or teniposide.

In some embodiments, the one or more mTOR (mammalian target of rapamycin, also known as the mechanistic target of rapamycin) inhibitors are selected from rapamycin, everolimus, temsirolimus and/or deforolimus.

In some embodiments, the one or more stilbenoids are selected from resveratrol, piceatannol, pinosylvin, pterostilbene, alpha-viniferin, ampelopsin A, ampelopsin E, diptoindonesin C, diptoindonesin F, epsilon-vinferin, flexuosol A, gnetin H, hemsleyanol D, hopeaphenol, trans-diptoindonesin B, astringin, piceid and/or diptoindonesin A.

In some embodiments, the one or more STING (Stimulator of interferon genes, also known as transmembrane protein (TMEM) 173) agonists may comprise cyclic di-nucleotides (CDNs), such as c-di-AMP, c-di-GMP, and cGAMP, and/or modified cyclic di-nucleotides that may include one or more of the following modification features: 2′-O/3′-O linkage, phosphorothioate linkage, adenine and/or guanine analogue, and/or 2′-OH modification (e.g. protection of the 2′-OH with a methyl group or replacement of the 2′-OH by —F or —N₃). In some embodiments, the one or more STING agonists are selected from BMS-986301, MK-1454, ADU-S100, a diABZI, 3′3′-cGAMP, and/or 2′3′-cGAMP.

In some embodiments, the one or more cancer vaccines are selected from an HPV vaccine, a hepatitis B vaccine, Oncophage, and/or Provenge.

In some embodiments, the one or more immunomodulatory agents may comprise an immune checkpoint inhibitor. The immune checkpoint inhibitor may target an immune checkpoint receptor, or combination of receptors comprising, for example, CTLA-4, PD-1, PD-L1, PD-L2, T cell immunoglobulin and mucin 3 (TIM3 or HAVCR2), galectin 9, phosphatidylserine, lymphocyte activation gene 3 protein (LAG3), MHC class I, MHC class II, 4-1BB, 4-1BBL, OX40, OX40L, GITR, GITRL, CD27, CD70, TNFRSF25, TL1A, CD40, CD40L, HVEM, LIGHT, BTLA, CD160, CD80, CD244, CD48, ICOS, ICOSL, B7-H3, B7-H4, VISTA, TMIGD2, HHLA2, TMIGD2, a butyrophilin (including BTNL2), a Siglec family member, TIGIT, PVR, a killer-cell immunoglobulin-like receptor, an ILT, a leukocyte immunoglobulin-like receptor, NKG2D, NKG2A, MICA, MICB, CD28, CD86, SIRPA, CD47, VEGF, neuropilin, CD30, CD39, CD73, CXCR4, and/or CXCL12.

In some embodiments, the immune checkpoint inhibitor is selected from urelumab, PF-05082566, MEDI6469, TRX518, varlilumab, CP-870893, pembrolizumab (PD1), nivolumab (PD1), atezolizumab (formerly MPDL3280A) (PD-L1), MEDI4736 (PD-L1), avelumab (PD-L1), PDR001 (PD1), BMS-986016, MGA271, lirilumab, IPH2201, emactuzumab, INCB024360, galunisertib, ulocuplumab, BKT140, bavituximab, CC-90002, bevacizumab, and/or MNRP1685A.

In some embodiments, the one or more immunomodulatory agents may comprise a complement pathway modulator. Complement pathway modulators modulate the complement activation pathway. Complement pathway modulators may act to block action of the C3 and/or C3a and/or C3aR1 receptor, or may act to block action of the C5 and/or C5a and/or C5aR1 receptor. In some embodiments, the complement pathway modulator is a C5 complement pathway modulator and may be selected from eculizumab, ravulizumab (ALXN1210), ABP959, RA101495, tesidolumab (LFG316), zimura, crovalimab (RO7112689), pozelimab (REGN3918), GNR-045, SOBI005, and/or coversin. In some embodiments, the complement pathway modulator is a C5a complement pathway modulator and may be selected from cemdisiran (ALN-CC5), IFX-1, IFX-2, IFX-3, and/or olendalizumab (ALXN1007). In some embodiments, the complement pathway modulator is a C5aR1 complement pathway modulator and may be selected from ALS-205, MOR-210/TJ210, DF2593A, DF3016A, DF2593A, avacopan (CCX168), and/or IPH5401.

In some embodiments, the one or more immunomodulatory agents may comprise an anti-TNFα agent. In some embodiments, the anti-TNFα agent may be an antibody or an antigen-binding fragment thereof, a fusion protein, a soluble TNFα receptor (e.g. a soluble TNFR1 or soluble TNFR2), an inhibitory nucleic acid, or a small molecule TNFα antagonist. In some embodiments, the inhibitory nucleic acid may be a ribozyme, a small hairpin RNA, a small interfering RNA, an antisense nucleic acid, or an aptamer. In some embodiments, the anti-TNFα agent is selected from adalimumab, certolizumab pegol, etanercept, golimumab, infliximab, CDP571, and biosimilars thereof (such as adalimumab-adbm, adalimumab-adaz, adalimumab-atto, etanercept-szzs, infliximab-abda and infliximab-dyyb).

In some embodiments, the one or more immunomodulatory agents may comprise azithromycin, clarithromycin, erythromycin, levofloxacin and/or roxithromycin.

In some embodiments, the one or more antibiotics are selected from amikacin, gentamicin, kanamycin, neomycin, netilmicin, tobramycin, paromomycin, streptomycin, spectinomycin, geldanamycin, herbimycin, rifaximin, loracarbef, ertapenem, doripenem, imipenem, cilastatin, meropenem, cefadroxil, cefazolin, cefalotin, cefalothin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftaroline fosamil, ceftobiprole, teicoplanin, vancomycin, telavancin, dalbavancin, oritavancin, clindamycin, lincomycin, daptomycin, azithromycin, clarithromycin, dirithromycin, erythromycin, roxithromycin, troleandomycin, telithromycin, spiramycin, aztreonam, furazolidone, nitrofurantoin, linezolid, posizolid, radezolid, torezolid, amoxicillin, ampicillin, azlocillin, carbenicillin, cloxacillin, dicloxacillin, flucloxacillin, mezlocillin, methicillin, nafcillin, oxacillin, penicillin G, penicillin V, piperacillin, temocillin, ticarcillin, calvulanate, ampicillin, subbactam, tazobactam, ticarcillin, clavulanate, bacitracin, colistin, polymyxin B, ciprofloxacin, enoxacin, gatifloxacin, gemifloxacin, levofloxacin, lomefloxacin, moxifloxacin, nalidixic acid, norfloxacin, ofloxacin, trovafloxacin, grepafloxacin, sparfloxacin, temafloxacin, mafenide, sulfacetamide, sulfadiazine, silver sulfadiazine, sulfadimethoxine, sulfamethoxazole, sulfanamide, sulfasalazine, sulfisoxazole, trimethoprim-sulfamethoxazole, sulfonamideochrysoidine, demeclocycline, minocycline, oytetracycline, tetracycline, clofazimine, dapsone, dapreomycin, cycloserine, ethambutol, ethionamide, isoniazid, pyrazinamide, rifampicin, rifabutin, rifapentine, streptomycin, arsphenamine, chloramphenicol, fosfomycin, fusidic acid, metronidazole, mupirocin, platensimycin, quinupristin, dalopristin, thiamphenicol, tigecycyline, tinidazole, trimethoprim, and/or teixobactin.

In some embodiments, the one or more antibiotics may comprise one or more cytotoxic antibiotics. In some embodiments, the one or more cytotoxic antibiotics are selected from an actinomycin, an anthracenedione, an anthracycline, thalidomide, dichloroacetic acid, nicotinic acid, 2-deoxyglucose, and/or chlofazimine. In some embodiments, the one or more actinomycins are selected from actinomycin D, bacitracin, colistin (polymyxin E) and/or polymyxin B. In some embodiments, the one or more antracenediones are selected from mitoxantrone and/or pixantrone. In some embodiments, the one or more anthracyclines are selected from bleomycin, doxorubicin (Adriamycin), daunorubicin (daunomycin), epirubicin, idarubicin, mitomycin, plicamycin and/or valrubicin.

In some embodiments, the one or more anti-fungal agents are selected from bifonazole, butoconazole, clotrimazole, econazole, ketoconazole, luliconazole, miconazole, omoconazole, oxiconazole, sertaconazole, sulconazole, tioconazole, albaconazole, efinaconazole, epoziconazole, fluconazole, isavuconazole, itraconazole, posaconazole, propiconazole, ravusconazole, terconazole, voriconazole, abafungin, amorolfin, butenafine, naftifine, terbinafine, anidulafungin, caspofungin, micafungin, benzoic acid, ciclopirox, flucytosine, 5-fluorocytosine, griseofulvin, haloprogin, tolnaflate, undecylenic acid, and/or balsam of Peru.

In some embodiments, the one or more anti-helminthic agents are selected from benzimidazoles (including albendazole, mebendazole, thiabendazole, fenbendazole, triclabendazole, and flubendazole), abamectin, diethylcarbamazine, ivermectin, suramin, pyrantel pamoate, levamisole, salicylanilides (including niclosamide and oxyclozanide), and/or nitazoxanide.

In some embodiments, other active agents are selected from growth inhibitory agents; anti-inflammatory agents (including non-steroidal anti-inflammatory agents; small molecule anti-inflammatory agents (such as colchicine); and anti-inflammatory biologics that target for example TNF, IL-5, IL-6, IL-17 or IL-33); JAK inhibitors; phosphodiesterase inhibitors; CAR T therapies; anti-psoriatic agents (including anthralin and its derivatives); vitamins and vitamin-derivatives (including retinoids, and VDR receptor ligands); steroids; corticosteroids; glucocorticoids (such as dexamethasone, prednisone and triamcinolone acetonide); ion channel blockers (including potassium channel blockers); immune system regulators (including cyclosporin, FK 506, and glucocorticoids); lutenizing hormone releasing hormone agonists (such as leuprolidine, goserelin, triptorelin, histrelin, bicalutamide, flutamide and/or nilutamide); hormones (including estrogen); and/or uric acid lowering agents (such as allopurinol).

Unless stated otherwise, in any of the ninth to seventeenth aspects of the invention, the subject may be any human or other animal. Typically, the subject is a mammal, more typically a human or a domesticated mammal such as a cow, pig, lamb, sheep, goat, horse, cat, dog, rabbit, mouse etc. Most typically, the subject is a human.

Any of the medicaments employed in the present invention can be administered by oral, parenteral (including intravenous, subcutaneous, intramuscular, intradermal, intratracheal, intraperitoneal, intraarticular, intracranial and epidural), airway (aerosol), rectal, vaginal, ocular or topical (including transdermal, buccal, mucosal, sublingual and topical ocular) administration.

Typically, the mode of administration selected is that most appropriate to the disorder, disease or condition to be treated or prevented. Where one or more further active agents are administered, the mode of administration may be the same as or different to the mode of administration of the compound, salt, solvate, prodrug or pharmaceutical composition of the invention.

The dose of the compounds, salts, solvates or prodrugs of the present invention will, of course, vary with the disease, disorder or condition to be treated or prevented. In general, a suitable dose will be in the range of 0.01 to 500 mg per kilogram body weight of the recipient per day. The desired dose may be presented at an appropriate interval such as once every other day, once a day, twice a day, three times a day or four times a day. The desired dose may be administered in unit dosage form, for example, containing 1 mg to 50 g of active ingredient per unit dosage form.

For the avoidance of doubt, insofar as is practicable any embodiment of a given aspect of the present invention may occur in combination with any other embodiment of the same aspect of the present invention. In addition, insofar as is practicable it is to be understood that any preferred, typical or optional embodiment of any aspect of the present invention should also be considered as a preferred, typical or optional embodiment of any other aspect of the present invention.

### BRIEF DESCRIPTION OF THE DRAWINGS

Embodiments of the invention will now be described, by way of example only, with reference to the accompanying drawings, in which:

FIG. 1: Structures of MCC7840, MCC950 IZ1201 and IZ1438 and their photoproducts in the photolysed solution in methanol;

FIG. 2: In-gel fluorescence scanning showing hNLRP3 photolabeled with photoprobes IZ1201 or IZ1438 without or with excessive MCC950 or MCC7840;

FIG. 3: Rank Order Distribution of proteins identified in the gel band corresponding to hNLRP3;

FIG. 4: MS1 intensity values of intact and IZ1438-modified hNLRP3 peptide ¹⁹⁵TCESPVSPIK²⁰⁴ following recombinant hNLRP3 labeling with IZ1438 in competition with MCC7840;

FIG. 5: MS2 spectra for the intact or IZ1438-modified peptide TCESPVSPIK of hNLRP3;

FIG. 6: Confirmation of the presence of NLRP3 in the supernatant of over expressing HEK cells (A) and in the column elution fraction (B);

FIG. 7: Confirmation of the presence of NLRP3 in supernatant of over expressing HEK cells and absence in control non-transfected HEK cells using two different antibodies (A and B);

FIG. 8: Radioligand binding assay optimisation;

FIG. 9: Tissue linearity in the radioligand binding studies;

FIG. 10: Radioligand binding studies, background assessment using non-transfected HEK lysates;

FIG. 11: Binding saturation studies;

FIG. 12: ATP competition of radioligand binding;

FIG. 13: NLRP3 model with predicted ligand binding sites;

FIG. 14: NLRP3 model with the prediction for the most likely ligand binding site, overlaid with the X-ray crystallography structures of ADP for both NLRC4 and NOD2 structures;

FIG. 15: NLRP3 model with MCC950 modelled into the active site, with the sulfonyl urea group located between the Walker A motif and the His522 residue;

FIG. 16: NLRP3 model with a selection of mutations associated with Cryopyrin-associated periodic syndrome (CAPS) which were identified as being close to the binding site.

### EXAMPLES

### Example 1: Photoaffinity Labeling Mass Spectrometry (PALMS)

### In Summary

The aim of this study was to apply Photoaffinity Labeling Mass Spectrometry (PALMS) to validate the interaction of MCC7840 with human NLRP3 (hNLRP3) and to identify amino acid residues contributing to the MCC7840-binding site of hNLRP3.

As a person skilled in the art would know, PALMS uses an analog of a biologically active ligand (a photoaffinity probe), that bears photo-reactive and reporter functional groups. The photoaffinity probe is designed and synthesized based on structure-activity relationships of a parent molecule. It is important to establish that the incorporated photo-reactive and reporter functional groups do not significantly alter the binding affinity of the ligand to its receptor and its functionality, compared with the non-derivatized ligand. During PALMS, the photoaffinity probe is incubated with the recombinant protein target, and irradiated with UV light. Subsequent to the complex formation, UV-irradiation of the photo-reactive group generates a highly reactive chemical species (e.g. carbene, nitrene, or radical) that covalently crosslinks the photoaffinity probe to its macromolecular binding partner. The photo-crosslinked protein target can be tagged by click chemistry with a fluorescent or an epitope-tag (e.g. TAMRA, biotin) and then visualized by the reporter group using SDS-PAGE and in-gel fluorescence scanning or Western blotting. Covalent bond formation between the probe and the protein partner enables the subsequent identification of probe-modified peptides and amino acids in the binding pocket using LC-MS/MS. The functional selectivity of the photoaffinity labeling event can be monitored through the addition of competitors in a control sample.

Study Design

In the first step, the experimental conditions of the photolabeling of recombinant hNLRP3 using two phototosensitive probes was optimized. In the second step, the photolabeling of hNLRP3 was carried out using one of the two photosensitive probes, and the photolabeled peptide(s)/amino acid(s) identified by label-free quantitative LC-MS/MS analysis.

| | |
|---|---|
| BHB | b-hydroxybutyrate |
| CID | Collision-induced dissociation |
| DMSO | Dimethyl sulfoxide |
| FDR | False discovery rate |
| HCD | Higher-energy collisional dissociation |
| HPLC | High performance liquid chromatography |
| hNLRP3 | Human NACHT, LRR and PYD |
| | domains-containing protein 3 |
| IC₅₀ | Half maximal inhibitory concentration |
| kDa | kilo Dalton |
| LC | Liquid chromatography |
| LFQ | Label free quantification |
| MeOH | Methanol |
| MS | Mass spectrometry |
| N₂ | Azote |
| NaCl | Sodium chloride |
| NH₄CO₃ | Ammonium bicarbonate |
| PAL | Photoaffinity labeling |
| PALMS | Photoaffinity labeling mass spectrometry |
| PBS | Phosphate buffer solution |
| RT | Retention time |
| SDS | Sodium dodecyl sulfate |
| SDS-PAGE | SDS-polyacrylamide gel electrophoresis |
| Sf21 | Spodoptera frugiperda 21 |
| TAMRA | Tetramethylrhodamine |
| TFA | Trifluoroacetic acid |
| UV | Ultraviolet |

Method

Photoactivatable analogues of MCC7840 were designed and synthesized by based on the SAR of MCC7840. Two photoprobes, IZ1201 and IZ1438, that retained the biological hallmarks of the parent molecule MCC7840 (evaluated in a cellular IL-1β release assay) were chosen to perform photoaffinity labeling experiments on purified recombinant hNLRP3 (6His-SUMO-TEV-NLRP3 [125-1036]) produced in Sf21 cells. To ensure efficient photolabeling of hNLRP3, optimised conditions were chosen for further PAL-MS experiments: 30-min treatment with 25 μM IZ1438 with or without an excess of parent drug MCC7840 50 μM. After protein digestion, probe-labeled peptides were analyzed by label-free quantitative mass spectrometry (MS). Peptide adducts with mass shift of 438.1727 m/z were analyzed with the MaxQuant software followed by manual interpretation of CID fragmentation spectra.

Results
The minimalist bifunctional photo-crosslinker in the probes had almost no negative impact on target engagement compared to parent compounds, under cellular conditions as shown in an IL-1β release assay in THP-1 cells.
IZ1201 and IZ1438 are cell-permeable probes that can infer MCC7840-target interactions in live cells.
Upon UV-irradiation at 365 nm, IZ1201 and IZ1438 generate a carbene intermediate that subsequently rearranges into the ethylene product, or reacts with solvent molecules to form a highly stable C—O covalent bond with methanol or the ketone product.
IZ1201 and IZ1438 bind to recombinant hNLRP3 and their binding is inhibited by the parent compound MCC7840 as well as the NLRP3 specific inhibitor MCC950.
During the MS1 analysis, one modified peptide ¹⁹⁵TCESPVSPIK²⁰⁴ was identified with a characteristic mass shift of +438,1727 m/z corresponding to the IZ1438 molecular weight minus N₂.
The probe-modified peptide was not detected in the control sample and less abundant in the presence of MCC7840 (thus competing with IZ1438)
During MS2 analysis, the modified peptide was identified with a characteristic mass shift of +265,0582 m/z resulting from the cleavage of the probe attached to the peptide upon CID fragmentation.
MS2 analysis of the probe-modified peptide and its intact counterpart localized the site of the adduct of 265.0582 m/z to E¹⁹⁷.

### Conclusion (in Summary)

These results demonstrate that IZ1438 photolabels E¹⁹⁷ in hNLRP3 in a MCC7840 competitive manner.

In Detail

PALMS uses a photoaffinity probe (an analog of a biologically active ligand (small-molecule, peptide) that bears photo-reactive and reporter functional groups. The photoaffinity probe is designed and synthesized based on structure-activity relationships of a parent molecule. It is important to establish that the incorporated photo-reactive and reporter functional groups do not significantly alter the binding affinity of the ligand to its receptor and its functionality, compared with the non-derivatized ligand. During PALMS, the photoaffinity probe is incubated with the recombinant protein target, and irradiated with UV light. Subsequent to the complex formation, UV-irradiation of the photo-reactive group generates a highly reactive chemical species (e.g. carbene, nitrene, or radical) that covalently crosslinks the photoaffinity probe to its macromolecular binding partner. The photo-crosslinked protein target can be tagged by click chemistry with a fluorescent or an epitope-tag (e.g. TAMRA, biotin) and then visualized by the reporter group using SDS-PAGE and in-gel fluorescence scanning or Western blotting. Covalent bond formation between the probe and the protein partner enables the subsequent identification of probe-modified peptides and amino acids in the binding pocket using LC-MS/MS. The functional selectivity of the photoaffinity labeling event can be monitored through the addition of competitors in a control sample.

Materials and Methods

Materials

Recombinant hNLRP3 [6His-SUMO-TEV-NLRP3 (125-1036), molecular weight 116,929 Da] produced in Sf21 insect cell line was stored at −80° C. in 10 mM Tris-HCl (pH 8.0), 150 mM NaCl, 1 mM DTT until use. Two different batches were used in the study: batch 1 (0.46 mg/mL; 4 μM) and batch 2 (0.20 mg/mL; 2 μM). Photoprobes IZ1201 and IZ1438, and parent compounds MCC950 and MCC7840 were provided by Inflazome (Table A).

TABLE A
Characteristics of MCC7840 and MCC950 as well as
the two analogues IZ1201 and IZ1438.

| | | | | | LC-MS & ¹H- |
|---|---|---|---|---|---|
| Name | Batch code | MW | Formula | Amount | NMR purity |
| MCC7840 | 10041523 | 388.49 | C₁₉H₂₄N₄O₃S | 5 × 1 mg | >95% |
| MCC950 | 10019401 | 404.48 | C₂₀H₂₄N₂O₅S | 5 × 1 mg | >95% |
| IZ1201 | 10043442 | 455.20 | C₂₃H₂₉N₅O₃S | 2.43 mg | >95% |
| IZ1438 | 10043543 | 466.56 | C₂₃H₂₆N₆O₃S | 3 mg | 95% |

Photoaffinity Labeling of Recombinant Human NLRP

Recombinant human NLRP3 (4 μg of batch 1 or batch 2, 3.4 pmol, final concentration 0.68 μM) was separately incubated in phosphate buffer saline (PBS) with each of the photoprobes (IZ1201 or IZ1438) at the indicated concentrations (diluted from DMSO stocks whereby DMSO never exceeded 1% in the final solution) or DMSO in 96-well plates (final reaction volume, 50 μL). After incubating in the dark at room temperature for 30 min, the mixture was photo-irradiated with UV light at 365 nm for 20 min at 4° C. For competitive photoaffinity labeling experiments, a 15 min pre-treatment with the parent compound MCC950 or MCC7840 at the indicated concentrations was followed by photoprobe treatment and photolysis. After UV-irradiation, 1% SDS and 10 mM DTT was added, and after incubation for 1 h at 56° C., protein samples were treated with 30 mM iodoacetamide for 45 min at room temperature in the dark. Probe-labeled hNLRP3 was tagged with tetramethylrhodamine (TAMRA) azide (100 μM TAMRA azide from 1 mM stock solution) by copper click chemistry using the Click-iT™ Protein Reaction Buffer Kit (ThermoFisher Scientific) according to the manufacturer's instructions. Dry acetone (9 volumes) pre-chilled to −20° C. was added and the cloudy mixture was vortexed thoroughly and incubated at −20° C. overnight. After centrifugation (15,000×g for 10 minutes at 4° C.), the supernatant was poured off and the remaining pellet washed with −20° C. acetone. The wash supernatant was removed by centrifugation and the precipitated protein pellet was air-dried for 10 min at room temperature.

Gel-Based Analysis of Crosslinked Proteins

Dry pellets of hNLRP3 (4 μg, 3.4 pmol) previously photolabeled with IZ1201 or IZ1438 with or without an excess of the parent compound MCC950 or MCC7840 were resuspended in 50 μL SDS loading buffer (Bio Rad's XT Sample Buffer containing 2.5% v/v 2-mercaptoethanol) and heated (60° C., 30 min). Proteins were resolved using SDS-PAGE (4-15% Criterion™ TGX Stain-Free™ Protein Gel, Bio Rad) and analyzed by in-gel fluorescence scanning using a ChemiDoc™ MP Imaging System (Bio Rad) with a green LED light as an excitation source and a BP600/20 nm emission filter. After in-gel fluorescence scanning, gels were stained with Coomassie blue to ensure the same amount of protein sample was loaded in each lane and imaged with the ChemiDoc™ MP Imaging System. Photoincorporation of each photoprobe in hNLRP3 was quantitatively assessed by measuring the fluorescent intensity of the corresponding gel band using ImageJ 1.52e and normalizing this value against the intensity value of hNLRP3 gel band stained with Coomassie blue to control for loading differences.

Preparation of Labeled hNLRP for MS-Analysis

Recombinant hNLRP3 (55 μg of batch 2, 47 pmol, final concentration 0.94 μM) in 50 μL phosphate buffer saline (PBS) was pre-incubated with 50 μM MCC7840 or vehicle for 15 min and then treated with 25 μM IZ1438 for further 30 min at room temperature. The samples were photo-irradiated for 20 min at 4° C. before quenching the photocrosslinking reaction with SDS loading buffer (4×stock, 17 μL). Proteins were resolved using SDS-PAGE (4-15% Criterion™ TGX Stain-Free™ Protein Gel, Bio Rad) and the gel was stained with Coomassie blue. Protein bands corresponding to hNLRP3 were cut out from the gel and washed for 2 h at 37° C. with 250 μl 50 mM NH₄HCO₃ and acetonitrile (ACN) (1:1) until Coomassie blue is removed. Thereafter, the gel pieces were treated at 56° C. for 30 min with 10 mM DTT in 50 mM NH₄HCO₃ and washed twice with 50 mM NH₄HCO₃ and ACN (1:1). This is followed by treatment with 55 mM iodoacetamide in 50 mM NH₄HCO₃ for 35 min at room temperature, washed twice with 50 mM NH₄HCO₃ and ACN (1:1), dried in a SpeedVac concentrator and rehydrated in 60 μL 50 mM NH₄HCO₃ solution containing 3 μg Trypsin/Lys-C Mix, Mass Spec Grade (Promega). The above mixture was incubated overnight for digestion at 37° C. under gentle agitation in the dark. After digestion, a short spin for 10 min was given and the “Trypsin/Lys-C fraction” was collected in fresh Axygen™ MaxyClear Snaplock Microtubes (ThemoFisher Scientific). The gel pieces were re-extracted twice with 100 μL 0.2% formic acid and ACN (1:1) and once with 50 μL ethanol and ACN (1:1) for 15 min with frequent vortexing. The supernatants were combined together with the “Trypsin/Lys-C fraction”, concentrated to dryness using a SpeedVac concentrator. Peptides (final concentration 0.55 μg/μL) were reconstituted in 100 μL 0.2% formic acid and 0.3% ACN in water and stored at −20° C. until analysis by LC-MS/MS.

Mass Spectrometry Analysis of Peptide Mixtures

Peptide mixtures were analyzed by nanoLC-MS/MS using a nanoAcquity UPLC (Waters) coupled to a QExactive HF mass spectrometer (Thermo Scientific) equipped with a nanoelectrospray source. Samples were diluted in 0.2% formic acid and 0.3% ACN in water to a final concentration of 0.05 μg/μl. The sample (1 μg, 20 μL) was loaded onto a C18 precolumn (Symmetry C18 NanoAcquity, 100 Å, 5 μm, 180 m×20 mm) at 20 μl/min in 0.2% formic acid and 0.3% ACN in water. After a desalting step (3 min), the precolumn was switched online with the analytical BEH C18 column (130 μm; 1.7 μm, 75 μM×250 mm, Waters) equilibrated in 92% solvent A (0.2% formic acid in water) and 8% solvent B (0.2% formic acid and 90% ACN in water). The XCalibur software controlled the MS and chromatography functions. The peptides were eluted using an 8-35% gradient of solvent B during 165 min at 270 nL/min flow rate. The mass spectrometer was operated in the data-dependent acquisition mode to automatically switch between MS and MS/MS acquisition. Survey full scan MS spectra (from m/z 325-1300) were acquired with a resolution of 60,000 at m/z 200. The AGC was set to 3×10⁶ with a maximum injection time of 45 ms. The top 20 most intense ions were targeted for fragmentation by higher-energy collisional dissociation (HCD) with normalized collision energy of 26% (AGC of 1×10⁵ and a maximum injection time of 60 ms for an intensity threshold of 3.3×104). The dynamic exclusion time window was set to 30 s to prevent repetitive selection of the same peptide. MS/MS spectra were recorded in profile type with a resolution of 15,000.

MS Data Processing

The raw files were processed with the MaxQuant software (version 1.5.3.8) (1) for peptide and protein identification and quantification. MS/MS raw files of the tryptic digests were searched using the Andromeda search engine against a concatenated database containing the human NLRP3 truncated sequence (125-1036) and the Spodoptera frugiperda (Sf21) database using the following parameters: carbamidomethylation of cysteine was set as fixed modification whereas N-terminal acetylation and methionine oxidation were set as variable modifications. All peptides were required to have a minimum peptide length of five amino acids and a maximum of two miss cleavages. Strict specificity for trypsin cleavage was required allowing cleavage of N-terminal to proline. The mass tolerances were set to 4.5 ppm and 20 ppm in MS and MS/MS respectively. The search was performed against a concatenated target-decoy database with modified reversing of protein sequences as described previously (2). The false discovery rate (FDR) for protein and peptide identifications was set to a maximum of 1%. To validate and transfer identifications across different runs, the ‘match between runs’ option in MaxQuant was enabled with a Match time window of 0.7 min and an Alignment time window of 20 min. Unknown modifications were identified by the “dependent peptides” setting implemented in MaxQuant in a standard search. The implemented algorithm performs spectrum matching to identify modified peptides in an unbiased manner. If an unidentified spectrum matches an identified spectrum, the mass shift (corresponding to the modification of the peptide) of the theoretical and observed precursor mass and the matched sequence will be reported. Modified peptides will be only identified if they are derived from an already identified unmodified peptide with a FDR of 1% and a mass tolerance of 6.5 mDa. Modified peptides were extracted from allPeptides.txt along with the ΔM mass shift between base and dependent peptides. All amino acids were considered as possible residues for modification. The mass of the modification used to search for probe-modified peptides was +438.17256 m/z for IZ1438, which is the mass for the corresponding probe minus a molecular nitrogen. This modification was set as a variable modification in all MaxQuant searches. For quantification purposes, label-free quantification (LFQ) intensities calculated by MaxQuant were used. The LFQ metric is derived from the raw intensities by the MaxLFQ algorithm, which uses a specific normalization procedure, as well as a particular aggregation method to calculate protein intensities, by taking into account, for each protein, all the peptide ratios measured in all pairwise comparisons of the different quantified samples (3). For LFQ quantification, only protein ratios calculated from at least two unique peptide ratios (min LFQ ratio count=2) were considered for calculation of the LFQ protein intensity. Analysis of the MaxQuant processed data was performed manually. In brief, for “dependent peptides” analysis, the “all.peptides.txt” file was opened in Excel and filtered for DP Proteins “sp|NRLP3-EV6347|”, DP Mass Difference “400<X<460” and DP Score “>60”. Selected peptides with a DP mass shift of +438.17256 m/z (with a tolerance of 5 ppm) and which are only present in the two conditions “NLRP3+IZ1438” and “NLRP3+IZ1438+MCC7840” and absent in the control “NRLP3” were considered as positive hits. Validation of the positive hits was carried out manually. MS spectra were visualized with the Xcalibur software to check the presence of the unmodified and modified peptides. Ideally, the unmodified peptide should be detected in all three conditions whereas the peptide modified with a +438.17265 m/z photoadduct should be detected in the condition “NLRP3+IZ1438” and to a lesser extent in the condition “NLRP3+IZ1438+MCC7840” but not in the control “NLRP3”. MS/MS spectra were visualized using the viewer program of MaxQuant to annotate y and b ions of the unmodified peptide. MS/MS spectra of the unmodified and modified peptides of interest (DP base scan and DP modif scan, respectively) were opened by Xcalibur software and the sequences of both peptides were compared to determine the position of the photoadduct in the sequence. A shift of +438.17265 m/z (with a tolerance of 5 ppm) on a y and/or a b ion is expected.

LC-MS/MS Analysis of Photolysis Products in Methanol

The photolysis of diazirine probes IZ1201 and IZ1438 in methanol was examined separately by analyzing the photoproducts produced using LC-MS/MS. Photoprobes (70 pmol/μL in MeOH) were kept in the dark or photo-irradiated at 365 nm for 20 min at 4° C. and then diluted 140 fold in 0.05% Trifluoroacetic acid (TFA) and 0.2% ACN in water to a final concentration of 500 fmol/μL. Photoprobe solutions were analyzed by nanoLC/MS-MS using an Ultimate 3500 RSLC System (Dionex) couple to an Orbitrap Velos Elite (Thermo Fisher Scientific) equipped with a nanoelectrospray source. Twenty μl of diluted photoprobe solution (10 pmol) was loaded onto a C-18 precolumn (Acclaim Pep Map C18, 100 Å, 5 μm, 300 μm×5 mm) at 20 μl/min in 0.05% TFA and 2% ACN in water. After a desalting step (3 min), the precolumn was switched online with the analytical BEH C18 column (130 μm; 1.7 μm, 75 μM×250 mm, Waters) equilibrated in 97% solvent A (0.2% formic acid) and 3% solvent B (0.2% formic acid and 80% ACN in water). Probes were eluted by a 3-99% gradient of solvent B during 13 min at a flow rate of 0.250 nl/min using a nano-HPLC system (U3000, Thermo Fisher Scientific) and directly electrosprayed via a nanoelectrospray ion source into an Orbitrap Velos Elite. The XCalibur software controlled the MS and chromatography functions. The mass spectrometer was operated in the data-dependent acquisition mode to automatically switch between MS and MS/MS acquisition. Survey full scan MS spectra (from m/z 100-1,600) were acquired with a resolution of 120,000. The AGC was set to 1×10⁶ with a maximum injection time of 200 ms. The top 7 most intense ions were targeted for fragmentation by collision-induced dissociation (CID) with normalized collision energy of 28% (AGC of 1×10⁵) and a maximum injection time of ms. Isolation windows at 2 m/z. The dynamic exclusion time window was set to 60 s to prevent repetitive selection of the same peptide. The relative abundance of the different species observed before and after photolysis was quantified from the MS ion intensity (or peak area) measured for each species. The percent composition of each component in the mixture was calculated based on MS ion intensity values.

Results

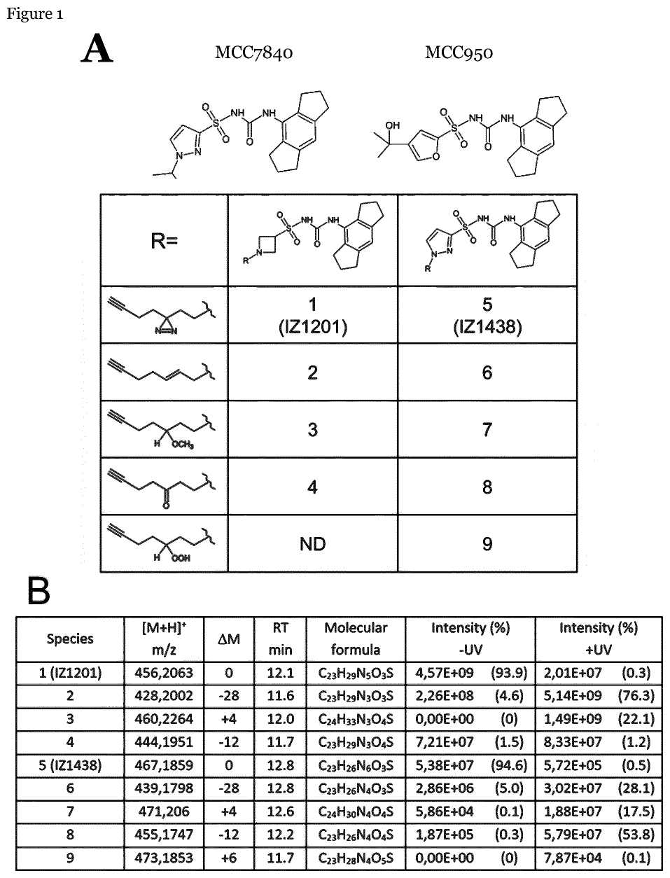

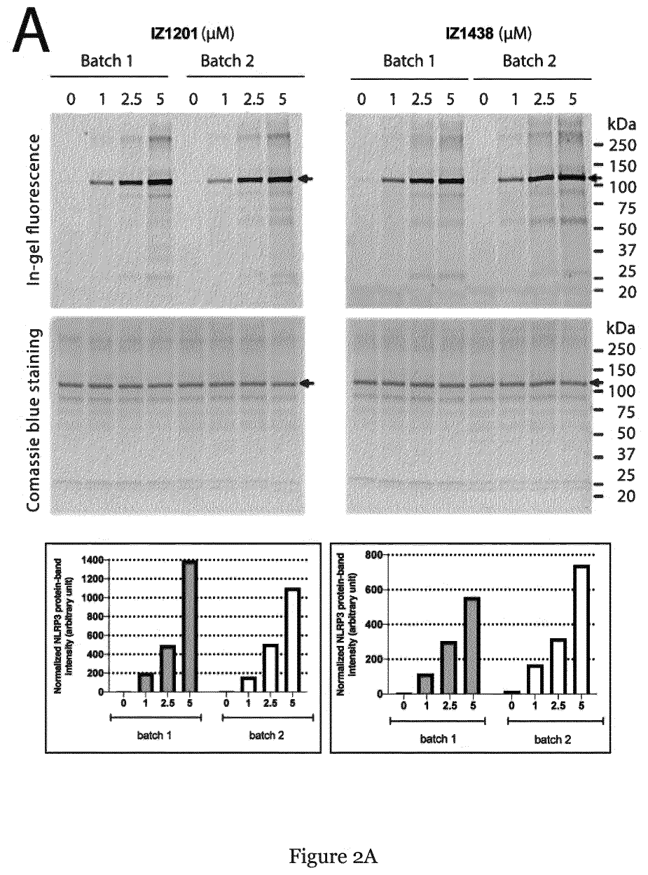

Two photoactivatable analogs of MCC7840 that contained both a photo-reactive crosslinking and a sorting functionality were designed and synthesized by Inflazome: FIG. 1: Structures of MCC7840 and MCC950, IZ1201 and IZ1438 and their photoproducts in the photolysed solution in methanol. (A) Structures (B) masses of molecular ions observed in the LC-MS mode, molecular formula, and molecular mass shifts compared to the probe prior photolysis. The relative abundance of the different species observed before and after photolysis was quantified from the MS ion intensity measured for each species. The percent composition of each component in the mixture was calculated based on MS ion intensity values.

An aliphatic diazirine moiety was chosen as the photocrosslinking group, owing to its small size (to minimize interference with protein binding) and short irradiation time needed to generate the highly reactive carbene intermediate upon photolysis. We used a small aliphatic alkyne reporter group, which can be conjugated to suitable reporter tags (fluorescent or biotin azide groups) using well-established bioorthogonal click chemistry for subsequent ex vivo PD/target identification by LC-MS/MS or dynamic cellular imaging of probe target complexes. The minimalist terminal alkyne-containing diazirine photo-crosslinker, previously described by L1 et al. 2013 (4), was incorporated in close proximity to the pharmacophore, maximizing the chance that on formation of the highly reactive carbene, the photo-reactive moiety reacts preferentially with the binding partner and not with the solvent.

To carry out PALMS experiments, it is necessary to choose a photoactivatable analogue of MCC7840 that retains the biological hallmarks of the parent molecule MCC7840 and has a mode of action and intracellular molecular interactions similar to MCC7840. In this purpose, the potencies (IC₅₀ values) and inhibitory effects of MCC7840, MCC950 and the two photoprobes IZ1201 and IZ1438 were assessed in an IL-1β release assay in THP-1 cells.

The inflammasomes function to activate caspase 1, which is then responsible for proteolytically cleaving and activating the pro-inflammatory cytokines interleukin-1β (IL-1β) and IL-18. Inflammasomes further promote inflammation by eliciting pyroptosis, a pro-inflammatory form of cell death. An IL-1β release assay in THP-1 cells was used to assess the ability of the different molecules to inhibit inflammasome-mediated cytokine secretion.

As shown in Table B, MCC950 is the most potent compound among the four tested while MCC7840 and photoprobe IZ1438 have comparable IC₅₀ values to each other, 4-6 fold lower than MCC950. Photoprobe IZ1201 is approximately 6-fold weaker in activity than IZ1438.

These results indicate that the minimalist bifunctional photo-crosslinker in the probes have minimal, to almost no, negative impact on target engagement compared to parent compounds, under cellular conditions.

TABLE B
IC₅₀ Values of MCC7840, MCC950 and photoactivatable analogs
IZ1201 and IZ1438 for inhibition of release of IL-1β from THP-1
cells following stimulation with LPS and nigericin.

| | MCC7840 | MCC950 | IZ1201 | IZ1438 |
|---|---|---|---|---|
| Assay | (IC₅₀ in nM) | (IC₅₀ in nM) | (IC₅₀ in nM) | (IC₅₀ in nM) |
| IL-1β release | 110 | 27 | 950 | 150 |

Photoaffinity Labeling of Recombinant Human NLRP3 and in Gel-Fluorescence Analysis

To validate the direct interaction between photoprobes and hNLRP3, we performed in vitro photoaffinity labeling experiments. Briefly, recombinant hNLRP3 from batch 1 or batch 2 was treated for 30 min with increasing concentrations of IZ1201 or IZ1438 followed by UV-irradiation to initiate photo-crosslinking. Subsequently, probe-labeled proteins were subjected to the click reaction through the aliphatic alkyne functional group on the probe with a red-fluorescent TAMRA azide dye so that the probe-labeled proteins(s) were selectively tagged with a TAMRA reporter fluorophore. Proteins were then resolved by SDS-PAGE followed by in-gel fluorescence scanning to visualize the fluorescent proteins.

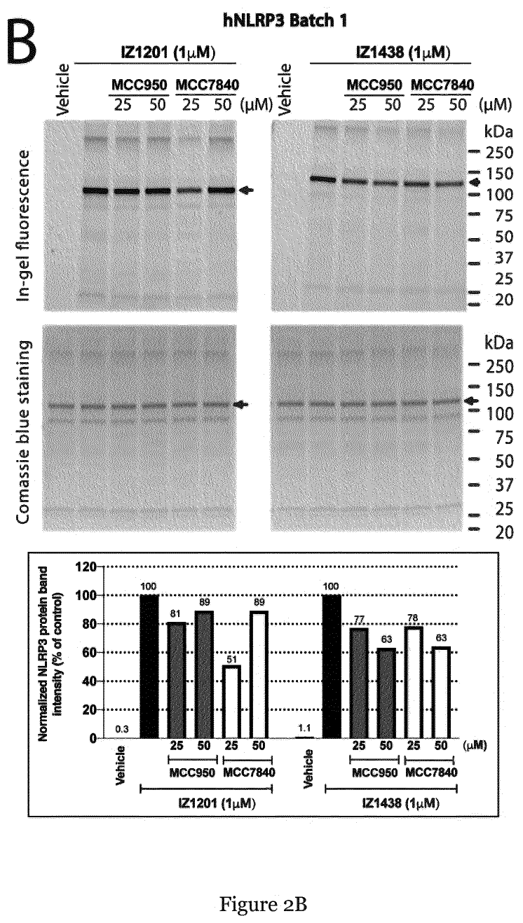

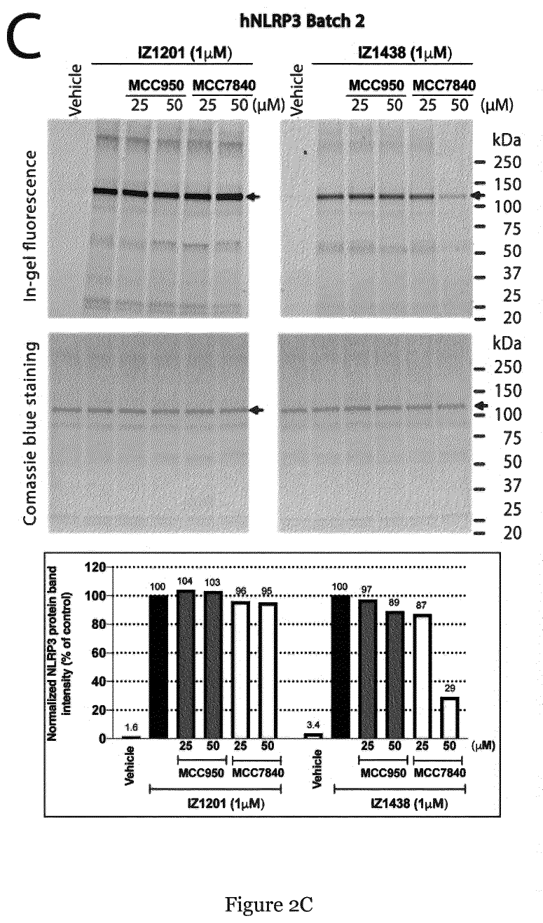

FIG. 2: In-gel fluorescence scanning showing hNLRP3 photolabeled with IZ1201 or IZ1438 without or with excessive MCC950 or MCC7840.

hNLRP3 was labeled with vehicle or indicated concentrations of IZ1201 or IZ1438 for 1 h followed by the standard photoaffinity labeling (PAL) procedure. Following photolysis, probe modified proteins were click-reacted with a TAMRA-azide tag and analyzed by SDS-PAGE and in-gel fluorescence scanning. For competition PAL experiments, hNLRP3 from batch 1 (B) or batch 2 (C) was pre-incubated for 15 min with MCC7840 or MCC950 (25 or 50 μM) or vehicle, then incubated for 1 h with or without IZ1201 or IZ1438 (1 μM) and this was followed by UV-irradiation, click-reaction with TAMRA-azide tag and in-gel fluorescence scanning as describe above. Photoincorporation of each photoprobe in hNLRP3 was quantitatively assessed by measuring the fluorescent intensity of the corresponding gel band (black arrow) and normalizing this value against the intensity value of hNLRP3 gel band stained with Coomassie blue.

As shown in FIG. 2A, similar labeling patterns were observed with both probes and for both hNLRP3 batches. We observed a dose-dependent photoincorporation of both probes into hNLRP3 (117 kDa). The yield of photo-incorporation was similar for both probes. Other protein bands (˜28, ˜60, ˜90 and ˜300 kDa seemed also to be labeled but to a much lesser extent than hNLRP3. A treatment with 1 μM of IZ1201 or IZ1438 provided sufficient labeling of hNLRP3 in vitro to envisage competition experiments with increasing concentrations of parent compounds MCC950 and MCC7840.

To explore the specificity of IZ1201 or IZ1438 labeling of hNLRP3, a set of competitive labeling experiments with MCC950 and MCC7840 were performed. In brief, hNLRP3 from batch 1 or batch 2 were pre-incubated for 15 min with MCC7840 or MCC950 (25 or 50 μM) or vehicle, then incubated for 1 h with IZ1201 or IZ1438 (1 μM) and this was followed by the standard photoaffinity labeling procedures. Proteins that are specifically labeled by the probes are those that exhibit a decrease in-gel fluorescent signal in samples pre-treated with parent compounds used as competitors. As shown in FIG. 2B, both MCC950 and MCC7840 weakly and rather inconsistently inhibited IZ1201 photoincorporation into hNLRP3 from batch 1 and batch 2. On the other hand, both competitors blocked IZ1438 labeling of hNRLP3 from batch 1 in a dose-dependent manner with similar potencies (˜23% inhibition at 25 μM and ˜37% inhibition at 50 μM). However, MCC950 weakly prevented the labeling of hNLRP3 from batch 2 by IZ1438 even at high dose (11% inhibition at 50 μM) whereas MCC7840 produced a dose-dependent inhibition of IZ1438 photoincorporation into hNLRP3 with a good potency (˜70% inhibition at 50 μM) (FIG. 2C).

Taken together, these data show that the two probes IZ1201 and IZ1438 bind to recombinant hNLRP3 and the parent compound MCC7840 blocks their binding as well as the NLRP3 specific inhibitor MCC950. We therefore conclude that IZ1201 and IZ1438 are viable photoaffinity probes to study the interaction of MCC7840 and analogs with hNLRP3. Further studies on the binding site of MCC7840 to hNLRP3 will be performed on hNLRP3 from batch 2 with IZ1438 as the selected probe and MCC7840 as the competitor.

Mapping IZ1438-Modified Peptides with Recombinant hNLRP3

To identify the exact residues photolabeled by IZ1438, hNLRP3 (batch 2, 0.94 μM) was photoirradiated alone or with IZ1438 (25 μM) in combination with or without MCC7840 (50 μM). After photolysis, samples were resolved using SDS-PAGE and proteins were stained with Coomassie blue. Protein bands corresponding to hNLRP3 were excised from the gel and subjected to in-gel trypsin proteolysis.

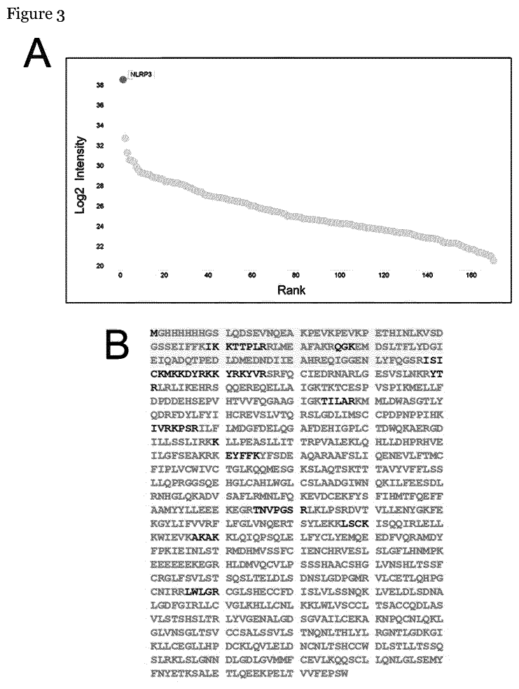

FIG. 3: Rank Order Distribution of proteins identified in the gel band corresponding to hNLRP3. A, The 172 proteins including hLNRP3 are respectively represented with red (hNLRP3) and blue (Sf21 proteins) circles. Proteins are ranked from the most (right) to the least (left) abundant. B, Sequence coverage diagram for 6His-SUMO-TEV-NLRP3 (125-1036). Peptides identified by LC-MS/MS are shown in red. The sequence of the 6His-SUMO-TEV tag is highlighted in yellow.

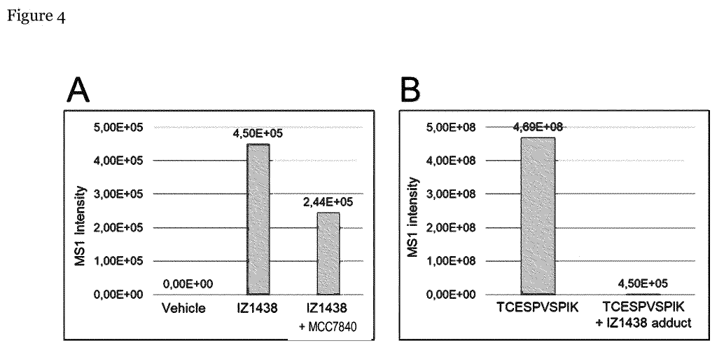

Overall, 172 proteins were identified including hNLRP3 as well as 171 Sf21 proteins. The rank order distribution of the 172 proteins based on their intensity is shown in FIG. 4A. Unsurprisingly, hNLRP3 is the most intense protein quantified in the gel bands. A sequence coverage of at least 90% for hNLRP3 was achieved for all samples (FIG. 3B).

The resulting peptides were analyzed by LC-MS/MS. MS data was searched by MaxQuant against a composite protein database including recombinant hNLRP3 and Spodoptera frugiperda protein sequences with the IZ1438 as a modification on any amino acid. Due to the nature of photochemical conjugation, a binding site may be represented by multiple conjugation events to several amino acid residues on one or more peptides. All peptide spectral matches (PSMs) assigned to a conjugated peptide were manually validated. Peptides with unknown modifications were identified using the “dependent peptides” setting implemented in MaxQuant in a standard search. This peptide adduct was also identified in the sample irradiated with the probe IZ1438 in the presence of the competitor MCC7840 but with a peak intensity 2 fold lower compared to the sample photolabeled with the probe alone. As expected, the precursor ion at 778.3711 m/z corresponding to the doubly charged signal from IZ1438-modified ¹⁹⁵TCESPVSPIK²⁰⁴ peak was not detected in the control sample (hNLRP3 UV-irradiated in the absence of IZ1438) (FIG. 4A). The base peak at 559.2817 m/z corresponding to the doubly charged intact peptide ¹⁹⁵TCESPVSPIK²⁰⁴ was −1,000 fold more intense than the corresponding probe-modified peptide indicating that the yield of specific covalent photoincorporation of IZ1438 in the binding site of hNLRP3 was low (FIG. 4B).

FIG. 4: MS1 intensity values of intact and IZ1438-modified hNLRP3 peptide: ¹⁹⁵TCESPVSPIK²⁰⁴ following recombinant hNLRP3 labeling with IZ1438 in competition with MCC7840. A unique tryptic peptide with the amino acid sequence TCESPVSPIK from hNLRP3 was detected by LC-MS/MS analysis with an increase in peptide mass of +438.1727 m/z corresponding to the incorporation of IZ1438 into this fragment. A, MS1 intensity value of the precursor ion at 778.3711 m/z (z=2) corresponding to IZ1438-modified TCESPVSPIK in the different samples. B, MS1 intensity values of precursor ions at 559.2817 m/z (z=2) and 778.3711 m/z (z=2) corresponding respectively to intact and IZ1438-modified TCESPVSPIK detected when hLNRP3 was labeled with IZ1438.

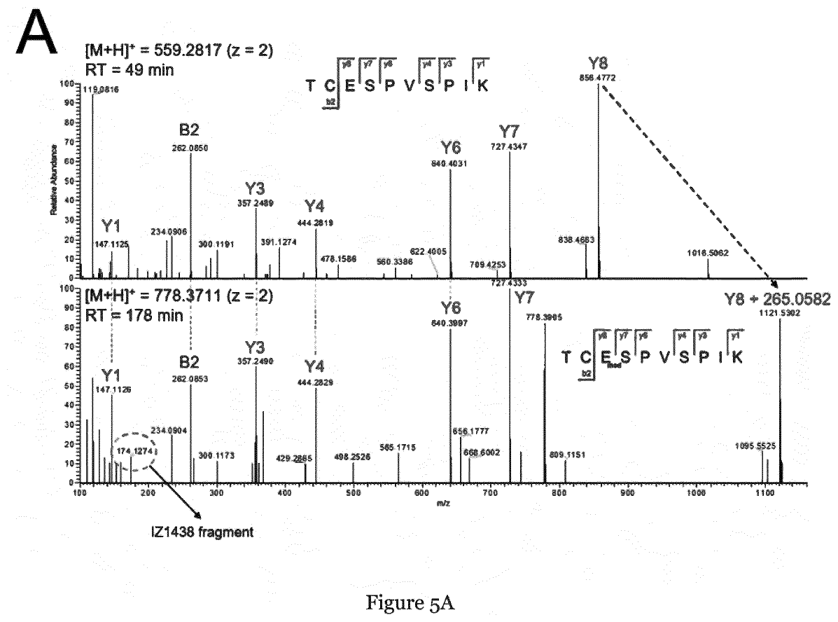

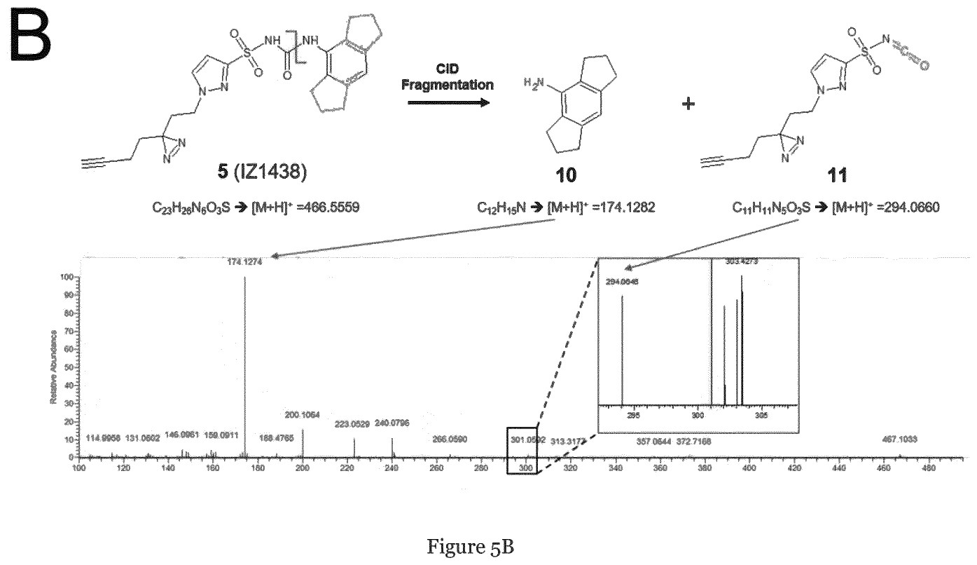

Examination of MS1 data showed that the IZ1438-modified ¹⁹⁵TCESPVSPIK²⁰⁴ fragment eluted later than the unmodified counterpart (178 min and 49 min, respectively), suggesting that following covalent attachment of IZ1438, the peptide adduct is more hydrophobic and therefore better retained on the C18 column. MS2 spectra of the probe-modified peptide (778.3711 m/z, z=2) and the unmodified form (559.2817 m/z, z=2) were manually evaluated for the presence of specific probe-labeled b- or y-type fragment ions and site localization of the photoadduct (to a specific amino acid residue). Both peptide forms shared several b- and y-type fragment ions, except for the y8 fragment ion which was detected with a mass of 856.4772 m/z in the MS2 spectrum of the probe-modified peptide and with a mass of 1121.5302 m/z in that of the unmodified peptide (FIG. 5A). This mass shift of +265.0582 m/z results from the cleavage of the probe attached to the peptide upon CID fragmentation.

Indeed, as shown in FIG. 5B, the CID fragmentation of IZ1438 in methanol generated two fragment ions, the hexahydro-s-indacen-4-amine 10 (174.1282 m/z) and the 1H-pyrazole-3-sulfonyl isocyanate 11 modified with the minimalist terminal alkyne-containing diazirine crosslinker (294.0661 m/z) resulting from the cleavage of the urea linkage. The mass of the adduct attached to the y8 fragment ion corresponds to the mass of 1H-pyrazole-3-sulfonyl isocyanate fragment containing the photo-crosslinker (294.0661 m/z) after loss of N₂. MS2 analysis of the probe-modified peptide and its intact counterpart localized the site of the adduct of 265.0582 m/z to E¹97. In addition, careful inspection of MS2 spectra also showed a fragment ion with a mass of 174.1126 m/z that was present only in the MS2 spectrum of the probe-modified peptide (FIG. 5A). This fragment ion likely corresponds to the hexahydro-s-indacen-4-amine which is released after photoadduct cleavage upon CID fragmentation. Our findings demonstrated that IZ1438 photolabeled, in an MCC7840-inhibitable manner, Glutamate 197 in hNLRP3.

FIG. 5: MS2 spectra for the intact or IZ1438-modified peptide TCESPVSPIK of hNLRP3: A, MS2 spectra of the probe-modified peptide 778.3711 m/z and its intact counterpart 559.2817 m/z. The y8 fragment ion of the probe-modified peptide carried the specific modification (+265.0582 m/z) corresponding to the adduct 11 derived from IZ1438 upon CID fragmentation and localized on E¹⁹⁷ (E_{mod}). In addition, the fragment ion 174.1126 m/z cleaved from IZ1438 was detected only in the MS2 spectrum of the probe-modified peptide. B, MS2 spectrum of IZ1438 showing specific daughter fragmentations 174.1274 m/z and 294.0646 m/z (enlarged MS2 spectrum)

### Conclusion

We successfully conducted PALMS on recombinant hLNRP3 using two novel photoaffinity probes IZ1201 and IZ1438 having a minimalist terminal alkyne-containing diazirine photo-crosslinker and showed that both active probes photolabel hNLRP3 in a protectable manner with MCC7840 and MCC950, potent and selective inhibitors of the NLRP3 inflammasome. These results show that MCC7840 and MCC950 bind hNLRP3 in vitro. Using PAL-MS with IZ1438 in competition with MCC7840, we identified the cross-linked amino acid E¹⁹⁷ as part of the binding site of MCC7840 in hNLRP3. To our knowledge, this is the first application of photoaffinity labeling on hNLRP3 to elucidate the cross-link position at an amino acid resolution by mass spectrometry. Our findings demonstrate the potential of chemical proteomics to map binding sites on hNLRP3 that interact with new inhibitors such as MCC7840.

Equipment
Spectramax Paradigm (Molecular devices)
PowerPac 200 (Bio-Rad)
Trans-Blot® Turbo™ Transfer System (Bio-Rad)
Centrifuge 1-15pk (Sigma)
ChemiDoc™ MP Imaging System (Bio-Rad)
Q-Exactive Plus (ThermoFisher Scientific)
nanoACQUITY UPLC system (Waters)
Ultimate 3500 RSLC System (Dionex)
Orbitrap Velos Elite (Thermo Fisher Scientific)
UVP CL-1000 UV crosslinking chamber (Hyland Scientific)

### REFERENCES

1. Cox J, Mann M. MaxQuant enables high peptide identification rates, individualized p.p.b.-range mass accuracies and proteome-wide protein quantification. Nat Biotechnol. 2008 December; 26(12):1367-72. doi: 10.1038/nbt.1511.
2. Elias J E, Gygi S P. Target-decoy search strategy for increased confidence in large-scale protein identifications by mass spectrometry/Nat Methods. 2007 March; 4(3):207-14.
3. Cox J, Hein M Y, Luber C A, Paron I, Nagaraj N, Mann M. Accurate proteome-wide label-free quantification by delayed normalization and maximal peptide ratio extraction, termed MaxLFQ. Mol Cell Proteomics. 2014 September; 13(9):2513-26. doi: 10.1074/mcp.M113.031591. Epub 2014 Jun. 17.
4. Li Z, Hao P, Li L, Tan C Y, Cheng X, Chen G Y, Sze S K, Shen H M, Yao S Q. Design and synthesis of minimalist terminal alkyne-containing diazirine photo-crosslinkers and their incorporation into kinase inhibitors for cell- and tissue-based proteome profiling. Angew Chem Int Ed Engl. 2013 Aug. 12; 52(33):8551-6. doi: 10.1002/anie.201300683.

### Example 2: Assessment of Compound Binding to HEK-NLRP3 Lysate Supernatants in a Competitive Radioligand Assay Format

The aim was to develop a radioligand binding assay utilising [H3]-MCC7840, and NLRP3 over-expressing HEK293 cell lysates. As NLRP3 is a cytoplasmic protein a conventional filtration binding assay method could not be used to separate free vs bound radiolabel from cell lysates. A gel filtration method was evaluated based on a literature method (Analytical Biochemistry 308, 2002 127-133) and the assay was optimised to evaluate tool compounds.

Assay Protocols

Supernatant Preparation

Cell pellets were defrosted over ice and diluted one in two with binding buffer. The resulting solution was aliquoted into 1.5 ml Eppendorf tubes and centrifuged (13.3 g×1000, 5 mins @ room temp). Supernatant was removed and stored at −20° C. Protein determination was performed on these samples using the Pierce BCA kit following the manufacturer's instructions.

Protein Isolation & Western Blotting

Cell supernatants were prepared in RIPA lysis buffer containing protease and phosphatase inhibitors and sonicated using single probe sonication. The BCA assay was used to determine protein concentration. Volumes of protein lysate containing equal amounts of protein were then separated on 4-12% Bis-Tris gels using sodium dodecyl sulfate-polyacrylamide gel electrophoresis (SDS-PAGE) and transferred to a nitrocellulose membrane using the iBLOT Gel Transfer system.

Blots were then blocked for 1 hour in odyssey blocking buffer, and then incubated overnight with primary antibody at 4° C. in Tris-buffered saline, 0.1% Tween 20 (TBST). Blots were then washed three times in TBST and incubated for 1.5 hours at room temperature with IRDye-conjugated goat anti-rabbit or anti-mouse IgG secondary antibody. Immunoreactive bands were visualized using the Odyssey L1-Cor InfraRed imaging system.

Radioligand Binding Assay

Final Assay Volume of 100 μl

Cell supernatant volume was dependent on protein concentration of each batch of cell supernatant.

10 ul 2 μM [3H]-MCC7840 (Final assay concentration (FAC)=200 nM) 10 ul Compound/Non Specific Binding (NSB) (10 uM MCC7840, FAC 10 uM)/DMSO control (FAC DMSO=0.1%)

Binding buffer volume was dependent on the volume of supernatant used in the assay. Samples were combined together and incubated, with shaking, for 4 hrs @ room temperature.

PD MultiTrap G-25 Preparation Plate (Final Gel Filtration Method)

Gel filtration separates molecules according to differences in size as they pass through a gel filtration medium packed in a column. The gel filtration medium is made up of spherical particles such as Sephadex with defined exclusion limits. As sample and buffer moves thorough the column and the molecules diffuse in and out of the pores. Smaller molecules move further into the pores so are retained longer in the column. Larger molecules cannot diffuse into the pores, so elute faster. Briefly, the PD MultiTrap plates were spun down at room temperature (RT), 800 g, for 1 minute to remove the storage buffer. They were washed five times with 300 μl/well binding buffer, 800 g, RT for 1 minute.

80 μl of sample was added per well, spun down at 400 g for 1 minute. 50 μl of the flow through was added to 145 μl Microscint-20 in Optiplates. The plate was shaken for approximately 30 minutes at RT prior to reading in a Perkin Elmer TopCount. Data was analysed using GraphPad Prism.

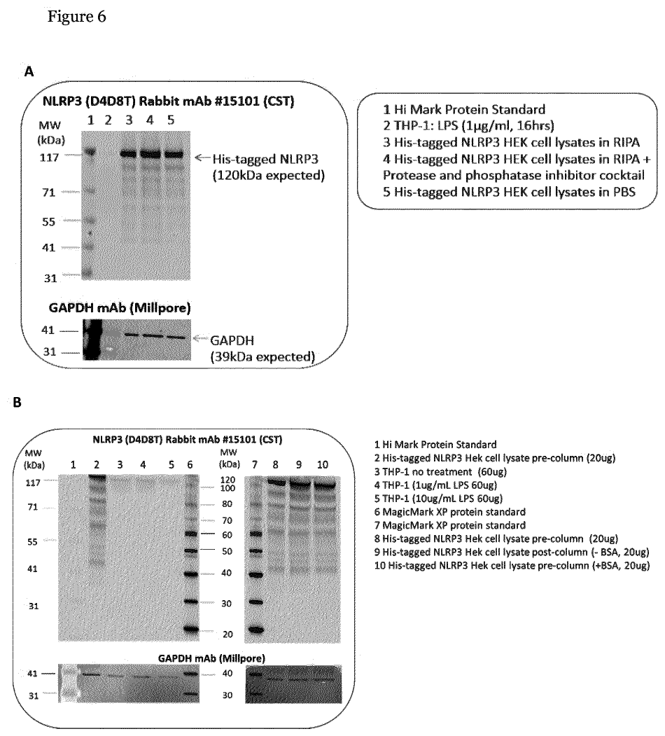

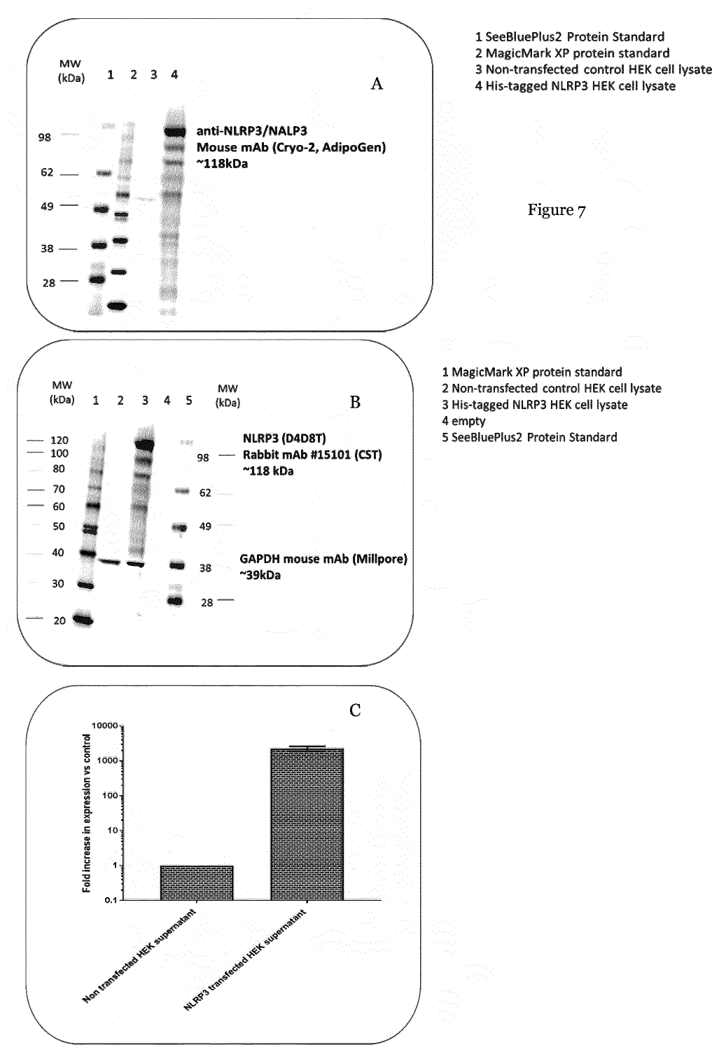

Confirmation of NLRP3 protein in the over-expressing HEK cell lysates NLRP3 expression was confirmed in the HEK293 cell lysates using western blotting as described above. The NLRP3 rabbit antibody from Cell Signalling Technologies (#15101) was used at 1:1000, GAPDH antibody was used at 1:5000 dilution, Alexa-fluor goat anti-rabbit 800 was used at 1:10000 dilution. The westerns were imaged using the Licor InfraRed imaging system. FIG. 6: Confirmation of the presence of NLRP3 in the supernatant of over expressing HEK cells (A) and in the column elution fraction (B). Multiple lysis buffers (PBS, RIPA with and without protease and phosphatase inhibitors) were compared and showed comparable results. Lysates from THP-1 cells stimulated with lipopolysaccharide were also compared on the same gel (A) but no band was detected in these samples. This may be due to the fact that a much lower amount of protein was extracted and loaded from these samples as shown by the lower intensity band detected for GAPDH. Higher amounts of protein loading showed a band corresponding to the correct size for NLRP3 (B) although this was not increased by lipopolysaccharide stimulation. HEK293-NLRP3 supernatants samples were compared prior to loading and from the elution fraction of the PD MultiTrap G-25 preparation Plate (B) for confirmation of the presence of NLRP3 in the eluate FIG. 7: Confirmation of the presence of NLRP3 in supernatant of over expressing HEK cells and absence in control non-transfected HEK cells using two different antibodies (A and B). Greater than 2000 fold increase in NLRP3 expression was detected in the transfected HEK cells compared to the non-transfected controls n=3 independent experiments (C).

Optimization of the PD MultiTrap™ G-25 Methods

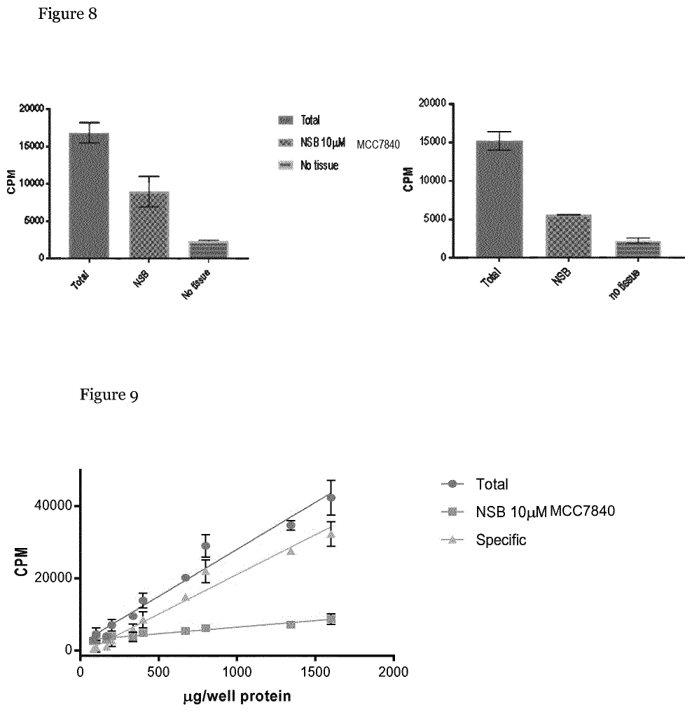

Separation of bound from free radiolabel was optimised through a series of experiments which investigated various centrifugation protocols and different buffer preparations. An attempt to move the samples by vacuum proved the columns too long to enable this procedure. However, by reducing the speed of the spin, it was possible to reduce the NSB and obtain an assay window of approximately three fold (FIG. 8: left hand graph: 800 g, 20 sec; right hand graph: 400 g, 1 min).

Tissue Linearity

Tissue linearity experiments were performed by varying the concentration of protein of the cell supernatants in the radioligand binding assay. Non-specific binding was defined using 10 μM of unlabeled compound MCC7840. The specific binding was determined by subtracting the non-specific binding from the total binding. The lowest concentration with a good assay window was determined to be 700 μg of protein per well. This was the protein concentration that was used in all subsequent experiments (FIG. 9: Radioligand binding studies (200 nM [³H]-MCC7840, 4 hrs @RT) Tissue linearity (n=3)).

Comparison of Non-Transfected Vs NLRP3 Transfected HEK Cell Supernatants

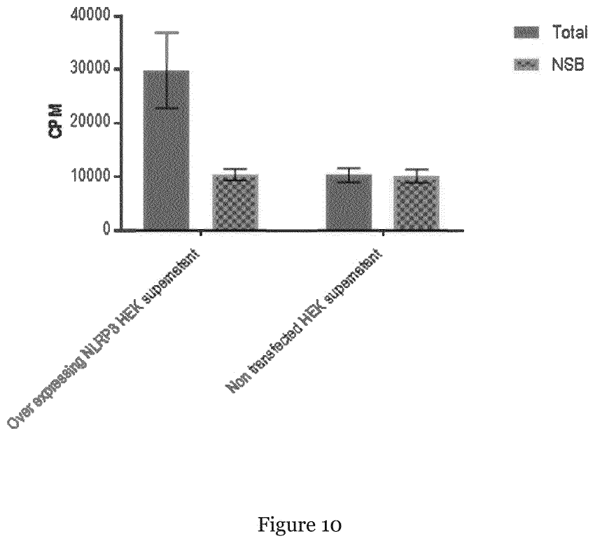

The assay signal was determined using non-transfected and NLRP3 transfected cell supernatants. Assessment of background signal was performed by comparing the total and non-specific binding in non-transfected HEK293 cell supernatants and NLRP3 over expressing cell supernatants in the assay as shown in FIG. 5. The total binding of 200 nM [³H]-MCC7840 was increased by approximately three fold in the NLRP3 cell supernatants compared to the non-transfected control supernatants (FIG. 10: Radioligand binding studies (700 μg protein, 200 nM [³H]-MCC7840, 4 hrs @RT)).

Radioligand Saturation Binding Studies

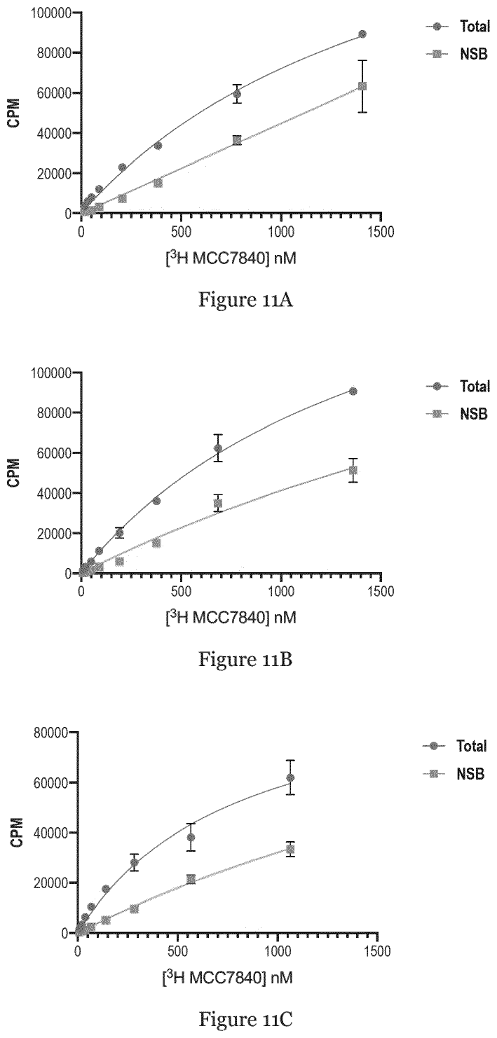

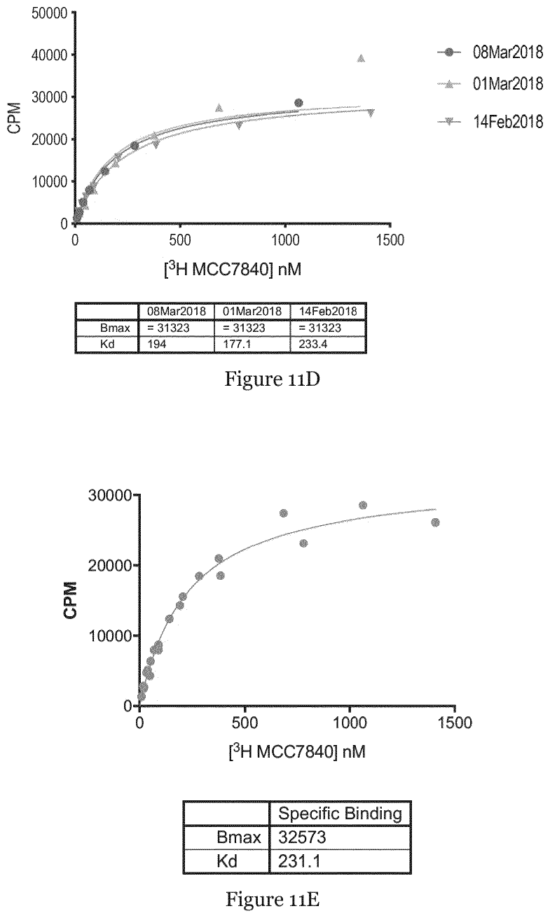

Saturation binding studies of [³H]-MCC7840 were performed by varying the concentration over a 200 fold range in three independent experiments to determine the Kd. All concentrations tested from the three separate experiments were combined to obtain a more accurate Kd (graph 6e). The Kd of [³H]-MCC7840 was determined to be approximately 230 nM from three independent experiments (FIG. 11).

Radioligand Competition Binding Studies with ATP and ADP

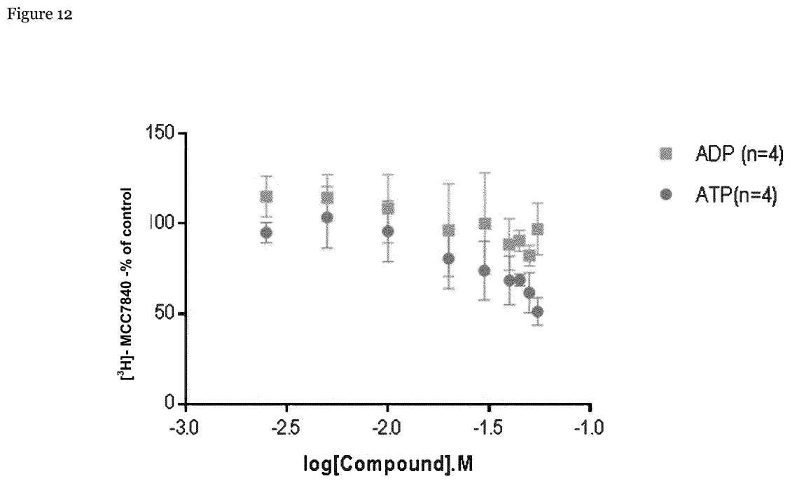

In order to illustrate whether the ligand binding is competitive with ATP and ADP experiments were performed by varying the concentration of ATP and ADP and competing with [³H]-MCC7840 at 200 nM. Although some competition was seen with ATP, higher concentrations could not be tested to define a full concentration response curve. An estimated IC₅₀ of 75 mM was obtained by constraining the minimum in the ATP curve fit (FIG. 12: Radioligand binding studies (700 μg protein, 200 nM [³H]-MCC7840, 4 hrs @RT)).

### Conclusions

The data presented in this report shows the successful development of a novel 96 well plate based gel filtration binding assay for the measurement of radioligand binding to NLRP3 in NLRP3 over-expressing HEK293 cell lysate supernatants. The assay was used to determine the binding characteristics of the NLRP3 radioligand [³H]-MCC7840.

| Reagents Reagent | Product Code | Supplier |
|---|---|---|
| HEK-NLRP3-FL cell lysate | | Inflazome |
| HEK non transfected cell lysate | | Inflazome |
| 96 well plates, flat bottom, clear | 3595 | Costar |
| PD MultiTrap ™ G-25 | 28-9225-26 | GE Healthcare Life |
| | | Sciences |
| TRIZMA HCl | T-3253 | SIGMA |
| NaCl₂ | J61807 | Alfa Aesar |
| KCl | P9333 | SIGMA |
| EDTA | E9884 | SIGMA |
| ATP | A2383 | SIGMA |
| ADP | A5285 | SIGMA |
| N-methyl-D-glucamine | M2004 | SIGMA |
| 96 well Optiplates | 6005290 | Perkin Elmer |
| MCC7840 10 mM stocks, DMSO | | Inflazome |
| [3H]-MCC7840 64.9 Cu/mmol, | 17-1204-1205 | Inflazome |
| 1 mCu/ml, 15.4 uM | | |
| Microscint-20 | 6013621 | Packard |
| 96 well, PP | 651201 | Greiner bio-one |
| Pierce ™ BCA Protein Assay Kit | 23227 | ThermoFisher |
| | | Scientific |
| Eppendorf 1.5 ml safe-lock ™ | T9661 | SIGMA-ALDRICH |

Binding Buffer Composition:

50 mM Tris HCl (7.88 g/l)

120 mM NaCl₂ (24 mls 5M/l)

5 mM KCl (0.372 g/l)

1 mM EDTA (0.292 g/l)

pH 7.4

### Example 3: Modelling

Digital constructs were created to provide a novel way to probe the NRLP3 protein, thereby giving mechanistic insight into the binding site of NLRP3 inhibitors.

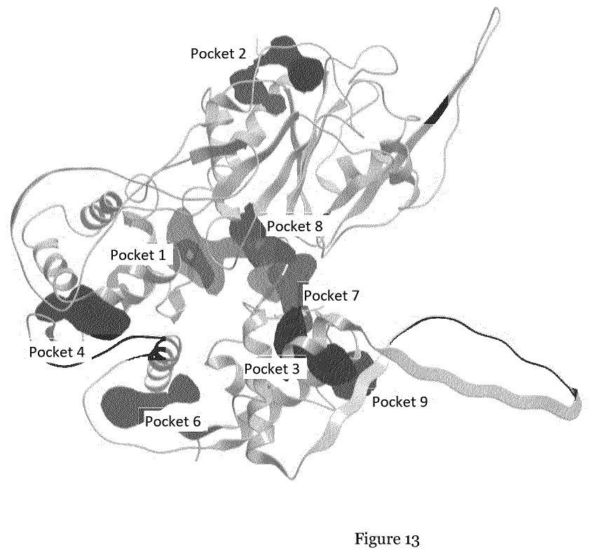

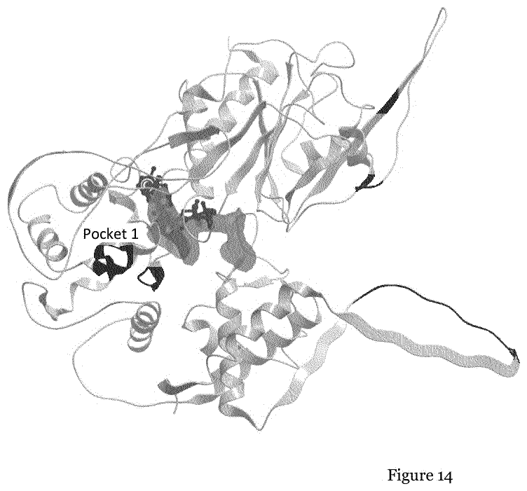

Multiple models of human NLRP3 were constructed from the X-ray crystal structure NACHT domains of mouse NLRC4 and rabbit NOD2 proteins (pdb codes 4kqv and 5irn respectively), using a manually constructed amino acid sequence alignment. These were analysed to identify the possible ligand binding sites (using an algorithm from MolSoft L.C.C): see FIG. 13, which shows one of the NLRP3 models, with predicted ligand binding sites. The largest and most likely binding site is Pocket 1, and consistently the most likely small molecule binding site is in an equivalent location as ADP from the crystal structures of NLRC4 and NOD2: see FIG. 14, which is an NLRP3 model with the prediction for the most likely ligand binding site, overlaid with the X-ray crystallography structures of ADP for both NLRC4 and NOD2 structures. The prediction for the most likely binding site encompasses the X-ray crystallography structure locations of the ADP molecules. The ATP binding site will have the same location.

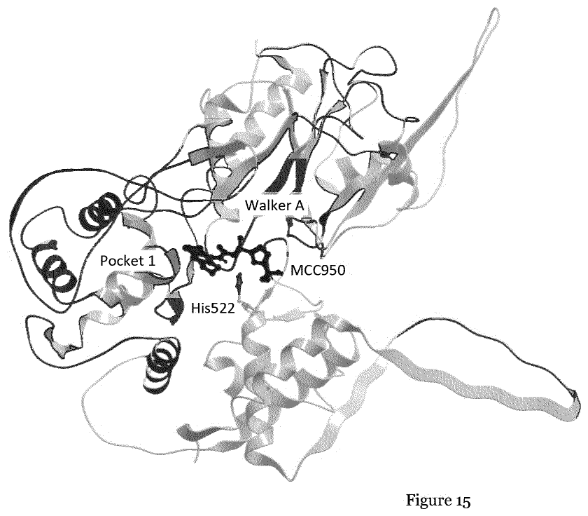

The X-ray crystal structures of NLRC4 and NOD2 show the Walker A motif binding a phosphate group, further stabilised by an adjacent histidine residue (His443 in NLRC4 and His583 in NOD2 structures). There is an equivalent histidine residue in human NLRP3, His522, and along with the Walker A binding motif, which defines an equivalent phosphate binding site for ATP/ADP in NLRP3. The small molecule inhibitor MCC950 contains a sulfonyl urea moiety, that mimics the phosphate group, and when modelled into the protein, positions the molecule to fill more of the space defined by pocket 1: see FIG. 15 which shows MCC950 modelled into the active site, with the sulfonyl urea group located between the Walker A motif and the His522 residue.

### Example 4: Mutagenesis Data

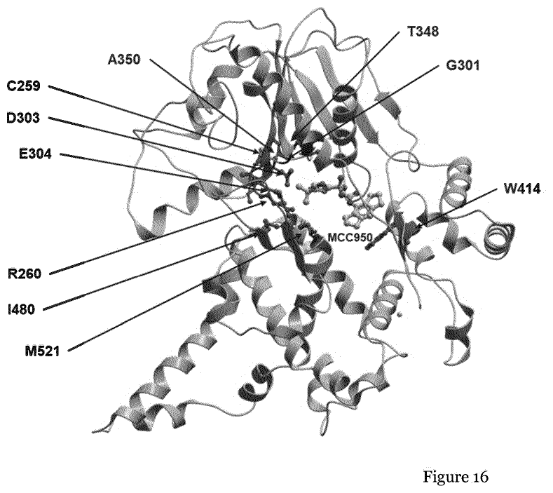

A selection of mutations associated with Cryopyrin-associated periodic syndrome (CAPS) were identified as being close to the active site of NLRP3: see FIG. 16 and Table C, below.

TABLE C
hNLRP3 clinical
variant number
T348M
G301D
W414L
M521T
I480F
R260W/L/P
E304K
D303N/A/H/G
C259W
A352V/T

In databases recording CAPs mutations (https://infevers.umai-montpellier.fr/web/search.php?n=4) coming from clinicians and some researchers, residue numbers are −2 amino acids from the protein sequence of NLRP3 in protein databanks like Uniprot (https://www.uniprot.org/uniprot/Q96P20). For the computational model in the present application, the reference sequence in Uniprot has been used. The only place where the clinical mutation annotations are included in the present application is Table C.

It is anticipated that one or more of the NLRP3 mutations detailed in Table D, below would prevent binding of NLRP3 inhibitors, render the NLRP3 protein inactive, render the NLRP3 protein constitutively active and/or provide structural insight into the binding pocket.

TABLE D
Human mutations
D302A
D305A
E306A
D302K
D305K
E306K
Q149A
C150A
E160A
W416A
V162A
S163A
S161A
E152A
D153A
R154A
N155A
A156G
R157A
L158A
G303A
G303R
G303H
G303K
D305R
D305K
D305E
E306D
E306R
E306H
Y565I
W416T
W416F
W416I

It will be understood that the present invention has been described above by way of example only. The examples are not intended to limit the scope of the invention. Various modifications and embodiments can be made without departing from the scope and spirit of the invention, which is defined by the following claims only.

1.-26. (canceled)

27. A binding site of the NLRP3 inflammasome, wherein the binding site:
(a) is at or proximal to the Walker A and/or Walker B site of the NLRP3 inflammasome; and/or
(b) comprises one or more residues selected from Arg183, Gly229, Ile230, Gly231, Lys232, Thr233, Ile234, Gly303, Asp305, Glu306, Leu413 and His522.

28. A method of inhibiting NLRP3 activation, the method comprising the step of binding a compound to the binding site of claim 27.

29. A method of treating a disease, disorder or condition responsive to NLRP3 inhibition, the method comprising the step of binding a therapeutically effective amount of a compound to the binding site of claim 27.

30. The method of claim 29, wherein the disease, disorder or condition is selected from:
(i) inflammation;
(ii) an auto-immune disease;
(iii) cancer;
(iv) an infection;
(v) a central nervous system disease;
(vi) a metabolic disease;
(vii) a cardiovascular disease;
(viii) a respiratory disease;
(ix) a liver disease;
(x) a renal disease;
(xi) an ocular disease;
(xii) a skin disease;
(xiii) a lymphatic condition;
(xiv) a psychological disorder;
(xv) graft versus host disease;
(xvi) pain;
(xvii) a condition associated with diabetes;
(xviii) a condition associated with arthritis;
(xix) a headache;
(xx) a wound or burn; and
(xxi) any disease where an individual has been determined to carry a germline or somatic non-silent mutation in NLRP3.

31. The method of claim 29, wherein the disease, disorder or condition is selected from:
(i) cryopyrin-associated periodic syndromes (CAPS);
(ii) Muckle-Wells syndrome (MWS);
(iii) familial cold autoinflammatory syndrome (FCAS);
(iv) neonatal onset multisystem inflammatory disease (NOMID);
(v) familial Mediterranean fever (FMF);
(vi) pyogenic arthritis, pyoderma gangrenosum and acne syndrome (PAPA);
(vii) hyperimmunoglobulinemia D and periodic fever syndrome (HIDS);
(viii) Tumour Necrosis Factor (TNF) Receptor-Associated Periodic Syndrome (TRAPS);
(ix) systemic juvenile idiopathic arthritis;
(x) adult-onset Still's disease (AOSD);
(xi) relapsing polychondritis;
(xii) Schnitzler's syndrome;
(xiii) Sweet's syndrome;
(xiv) Behcet's disease;
(xv) anti-synthetase syndrome;
(xvi) deficiency of interleukin 1 receptor antagonist (DIRA); and
(xvii) haploinsufficiency of A20 (HA20).

32. A method of reducing cellular or mitochondrial Reactive Oxygen Species (ROS) by inhibiting NLRP3 activation, the method comprising the step of binding a compound to the binding site of claim 27.

33. The method of claim 28, wherein the compound:
(i) is a small molecule, peptide, polypeptide, oligonucleotide, protein, antibody or aptamer; and/or
(ii) is adapted to bind covalently or non-covalently to the binding site; and/or
(iii) effects inhibition of activation of NLRP3 and thereby prevents ATP displacing ADP from the Walker A and/or Walker B site of NLRP3; and/or
(iv) effects inhibition of activation of NLRP3 by binding to one or more residues selected from Arg183, Gly229, Ile230, Gly231, Lys232, Thr233, Ile234, Gly303, Asp305, Glu306, Leu413 and His522; and/or
(v) comprises a motif that acts as a phosphonate mimic.

34. The method of claim 29, wherein the compound:
(i) is a small molecule, peptide, polypeptide, oligonucleotide, protein, antibody or aptamer; and/or
(ii) is adapted to bind covalently or non-covalently to the binding site; and/or
(iii) effects inhibition of activation of NLRP3 and thereby prevents ATP displacing ADP from the Walker A and/or Walker B site of NLRP3; and/or
(iv) effects inhibition of activation of NLRP3 by binding to one or more residues selected from Arg183, Gly229, Ile230, Gly231, Lys232, Thr233, Ile234, Gly303, Asp305, Glu306, Leu413 and His522; and/or
(v) comprises a motif that acts as a phosphonate mimic.

35. The method of claim 32, wherein the compound:
(i) is a small molecule, peptide, polypeptide, oligonucleotide, protein, antibody or aptamer; and/or
(ii) is adapted to bind covalently or non-covalently to the binding site; and/or
(iii) effects inhibition of activation of NLRP3 and thereby prevents ATP displacing ADP from the Walker A and/or Walker B site of NLRP3; and/or
(iv) effects inhibition of activation of NLRP3 by binding to one or more residues selected from Arg183, Gly229, Ile230, Gly231, Lys232, Thr233, Ile234, Gly303, Asp305, Glu306, Leu413 and His522; and/or
(v) comprises a motif that acts as a phosphonate mimic.

36. A method of screening a compound, the method comprising the steps of: (i) exposing the compound to the binding site of claim 27, and (ii) determining the extent of binding of the compound to the binding site.

37. The method of claim 36, wherein the extent of binding of the compound to the binding site is determined by mass spectrometry, NMR, X-ray crystallography, SPR or radioligand binding.

38. A method of screening a compound, the method comprising the steps of: (i) simulating on a computer exposing the compound to the binding site of claim 27, and (ii) determining the extent of binding of the compound to the binding site.

39. A compound identified by a method as claimed in claim 36, or a pharmaceutically acceptable salt, solvate or prodrug thereof.

40. A compound identified by a method as claimed in claim 38, or a pharmaceutically acceptable salt, solvate or prodrug thereof.

41. A compound adapted to bind to the binding site of claim 27, or a pharmaceutically acceptable salt, solvate or prodrug thereof.

42. A pharmaceutical composition comprising a compound or a pharmaceutically acceptable salt, solvate or prodrug as claimed in claim 39, and a pharmaceutically acceptable excipient.

43. A pharmaceutical composition comprising a compound or a pharmaceutically acceptable salt, solvate or prodrug as claimed in claim 40, and a pharmaceutically acceptable excipient.

44. A pharmaceutical composition comprising a compound or a pharmaceutically acceptable salt, solvate or prodrug as claimed in claim 41, and a pharmaceutically acceptable excipient.

45. A method of inhibiting NLRP3 activation, the method comprising the use of a compound or a pharmaceutically acceptable salt, solvate or prodrug as claimed in claim 39, to inhibit NLRP3 activation.

46. A method of inhibiting NLRP3 activation, the method comprising the use of a compound or a pharmaceutically acceptable salt, solvate or prodrug as claimed in claim 40, to inhibit NLRP3 activation.

47. A method of inhibiting NLRP3 activation, the method comprising the use of a compound or a pharmaceutically acceptable salt, solvate or prodrug as claimed in claim 41, to inhibit NLRP3 activation.
